(19) United States
(12) Patent Application Publication
(10) Pub. No.: US 2026/0108347 A1
(43) Pub. Date: Apr. 23, 2026

(54) ASSEMBLY FOR INTRAOCULAR LENS INJECTION DEVICE

(71) Applicant: ALCON INC., Fribourg (CH)

(72) Inventors: Nanhong LOU, Southlake, TX (US); Harlen HOANG, Fort Worth, TX (US); Mitchell SHERRY, Fort Worth, TX (US)

(21) Appl. No.: 19/364,206

(22) Filed: Oct. 21, 2025

Related U.S. Application Data
(60) Provisional application No. 63/710,103, filed on Oct. 22, 2024.

(51) Int. Cl. A61F 2/16

(52) U.S. Cl. CPC A61F 2/1667

(57) ABSTRACT

The present disclosure relates to an assembly operable with an intraocular lens (IOL) injection device. The assembly includes an electric motor and a control device. The electric motor is configured to generate rotational motion and includes a stator and a drive shaft. The stator or the drive shaft is removably couplable to an adjustment knob of the IOL injection device. The control device is communicatively coupled to the electric motor and is configured to control operation of the electric motor. The stator or the drive shaft is configured to rotate the adjustment knob relative to a body and a plunger rod of the IOL injection device in response to the rotational motion.

### CROSS-REFERENCE TO RELATED APPLICATION

This application claims priority to U.S. Provisional Patent Application No. 63/710,103, filed on October 22, 2024, the disclosure of which is incorporated herein by reference in its entirety.

### BACKGROUND

The human eye functions to provide vision by transmitting light through a clear outer portion called the cornea, and focusing the image by way of a crystalline lens onto a retina. The quality of the focused image depends on many factors including the size and shape of the eye, and the transparency of the cornea and the lens. When age or disease causes the lens to become less transparent, vision deteriorates because of the diminished light transmitted to the retina. This deficiency in the lens of the eye is medically known as a cataract. An accepted treatment for this condition is surgical removal of the native lens and replacement of the lens function by an artificial intraocular lens (IOL).

The IOL is injected into the eye using a delivery device. Several manual IOL injection devices are available for implanting these lenses into the eye. However, threaded-type manual IOL injection devices require the use of two hands, which may be cumbersome and tedious.

### SUMMARY

The present disclosure relates to an assembly operable with an intraocular lens (IOL) injection device. The assembly includes an electric motor and a control device. The electric motor is configured to generate rotational motion, the electric motor including a stator and a drive shaft, the stator or the drive shaft removably couplable to an adjustment knob of the IOL injection device. The control device is communicatively coupled to the electric motor and is configured to control operation of the electric motor. The stator or the drive shaft is configured to rotate the adjustment knob relative to a body and a plunger rod of the IOL injection device in response to the rotational motion. The assembly may further include a flex shaft cable having a first end coupled to the drive shaft of the electric motor and a second end couplable to the adjustable knob of the IOL injection device. The flex shaft cable is configured to transmit the rotational motion from the electric motor to the adjustment knob.

### BRIEF DESCRIPTION OF THE DRAWINGS

The present disclosure relates to systems and methods for performing registration of an eye based on transition edges within the eye, wherein:

FIG. 1 illustrates an example operating environment in which a motor assembly for an IOL injection device may be implemented;

FIG. 2 illustrates an example implementation of a phacoemulsification surgical console of FIG. 1;

FIG. 3 illustrates a schematic showing a fluidics subsystem and hand piece of the phacoemulsification surgical console of FIG. 1;

FIGS. 4A-4C illustrate various example motor assemblies that may be implemented in the environment of FIG. 1;

FIGS. 5A-5E illustrate an example IOL injection device to which a motor assembly may be attached;

FIGS. 6A and 6B are cross-sectional views of the IOL injection device of FIGS. 5A-5E with an adjustment knob and plunger rod at two different axial locations relative to a body of the IOL injection device;

FIG. 7 is a front perspective view of an example cartridge loaded in the IOL injection device of FIGS. 5A-5E;

FIG. 8 is a side view of the IOL injection device of FIGS. 5A-5C with an example motor assembly;

FIGS. 9A-9B illustrate example clamps to couple a motor assembly to an IOL injection device;

FIGS. 10A-10B include cross-sectional views of two example electric motor assemblies that may be coupled to an IOL injection device;

FIGS. 11A-11C illustrate an example hydraulic motor assembly that may be coupled to an IOL injection device;

FIG. 12 illustrates an example IOL injection system that includes an assembly operable with an IOL injection device; and

FIGS. 13A-13C illustrate various example IOL injection systems that each includes an assembly operable with an IOL injection device.

### DETAILED DESCRIPTION

The present disclosure is directed to, among other things, devices, systems, and methods to motorize a manual IOL injection device for singlehanded operation. Accordingly, some embodiments may upgrade a manual two-handed IOL injection device to an automatic singlehanded device. A surgeon may then use their free hand for any other tasks as needed during IOL delivery, such as holding a second tool to stabilize the patient’s eye, or the like.

Some single-use automatic IOL injection devices exist that use CO₂ cartridges to power injection of the IOL. Such IOL injection devices as well as the CO₂ cartridges that power them are disposable, which has the disadvantage of providing a single use per device and cartridge before adding materials to landfills. Embodiments herein, however, may pair a reusable or disposable motor assembly with a reusable IOL injection device to motorize or automate the IOL injection device without filling landfills after a single use. In addition, such motor assemblies may be added on to existing IOL injection devices surgeons already use and are familiar with to allow the surgeons to additionally obtain the benefit of automated/motorized functionality for singlehanded use. Providing such motor assemblies as add-ons to existing IOL injection devices may speed time to market of an automated motorized IOL injection device since the motor assemblies may not require as much time and hardware investment or testing and certification as the IOL injection devices themselves.

An example motor assembly for an IOL injection device may generally include a motor that is attached to the IOL injection device. The motor is attachable to a power supply (electrical or fluid) as well as a control device, either or both of which may be included as part of the motor assembly or separately therefrom. The motor includes a first component that is removably couplable to a first portion of the IOL injection device. The first component of the motor assembly may have a fixed angular orientation relative to the first portion of the IOL injection device when coupled to the first portion of the IOL injection device. The motor also includes a second component removably couplable to a second portion of the IOL injection device that is rotatable relative to the first portion of the IOL injection device. The second component may be drivable by the motor to rotate the second portion of the IOL injection device relative to the first portion of the IOL injection device, which in turn may axially translate a plunger rod of the IOL injection device.

As a more particular example, the motor may be an electric motor with a stator and a drive shaft. The “first component” of the motor mentioned previously may include the stator or the drive shaft. The “second component” of the motor mentioned previously may include the other of the drive shaft or the stator. The “first portion” of the IOL injection device mentioned previously may include a body or a plunger rod of the IOL injection device. The “second portion” of the IOL injection device mentioned previously may include an adjustment knob of the IOL injection device. Thus, in one example, the stator of the electric motor is removably coupled to the body of the IOL injection device with a fixed angular orientation relative to the body and the drive shaft is removably coupled to the adjustment knob with a fixed angular orientation relative to the adjustment knob. In operation, the stator is rotationally fixed relative to the body such that operation of the electric motor turns or rotates the drive shaft and thus the adjustment knob relative to the stator and the body, which in turn causes the adjustment knob to translate axially relative to the body, which in turn causes the plunger rod to translate axially relative to the body. In another example, the stator of the electric motor is removably coupled to the adjustment knob of the IOL injection device with a fixed angular orientation relative to the adjustment knob and the drive shaft is removably coupled to the plunger rod with a fixed angular orientation relative to the plunger rod. In operation, the drive shaft is rotationally fixed relative to the plunger rod and therefore the body such that operation of the electric motor turns or rotates the stator and thus the adjustment knob relative to the body, which in turn causes the adjustment knob to translate axially relative to the body, which in turn causes the plunger rod to translate axially relative to the body.

The control device in the case of the electric motor may include a button coupled to a potentiometer that controls rotational speed (and/or also turns on/off) the electric motor. The potentiometer may be wirelessly coupled or hardwired to the electric motor. Alternatively or additionally, the control device may include a foot pedal of a phacoemulsification surgical console to which the electric motor is coupled. The power supply may include a battery or the phacoemulsification surgical console.

As another particular example, the motor may be a hydraulic motor with a stator and a rotor. The “first component” of the motor mentioned previously may include the stator or the rotor. The “second component” of the motor mentioned previously may include the other of the rotor or the stator. The “first portion” of the IOL injection device mentioned previously may include a body or a plunger rod of the IOL injection device. The “second portion” of the IOL injection device mentioned previously may include an adjustment knob of the IOL injection device. Thus, in one example, the stator of the electric motor is removably coupled to the body of the IOL injection device with a fixed angular orientation relative to the body and the rotor is removably coupled to the adjustment knob with a fixed angular orientation relative to the adjustment knob. In operation, the stator is rotationally fixed relative to the body such that operation of the electric motor turns or rotates the rotor and thus the adjustment knob relative to the stator and the body, which in turn causes the adjustment knob to translate axially relative to the body, which in turn causes the plunger rod to translate axially relative to the body. In another example, the stator of the electric motor is removably coupled to the adjustment knob of the IOL injection device with a fixed angular orientation relative to the adjustment knob and the rotor is removably coupled to the plunger rod with a fixed angular orientation relative to the plunger rod. In operation, the rotor is rotationally fixed relative to the plunger rod and therefore the body such that operation of the electric motor turns or rotates the stator and thus the adjustment knob relative to the body, which in turn causes the adjustment knob to translate axially relative to the body, which in turn causes the plunger rod to translate axially relative to the body.

In the case of the hydraulic motor, a phacoemulsification surgical console may serve as the power supply (or specifically a fluid supply) for the hydraulic motor and the control device may include a foot pedal of the phacoemulsification surgical console. In this and other embodiments, the same input/output fluid lines of the phacoemulsification surgical console used with a phacoemulsification cutting tip to emulsify and remove a patient’s native eye lens (or alternatively redundant input/output fluid lines) may be coupled to the hydraulic motor to power the hydraulic motor for insertion of an artificial IOL after removal of the patient’s native lens.

Embodiments of the present disclosure may be utilized with any suitable system, apparatus or device that includes a threaded type IOL injection device, and particularly in some embodiments with manually operated threaded type IOL injection devices. Example IOL injection devices with which some embodiments herein may be implemented include the MONARCH III and MONARCH IV delivery systems available from ALCON but may alternatively or additionally be implemented with other IOL injection devices from ALCON and/or from other vendors/suppliers.

The embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise. Further, one or more of the figures and accompanying descriptions are given with respect to ophthalmic surgical systems. However, such uses are not meant to be limiting such that the devices described may be used in any number of different contexts and applications where it may be helpful or applicable.

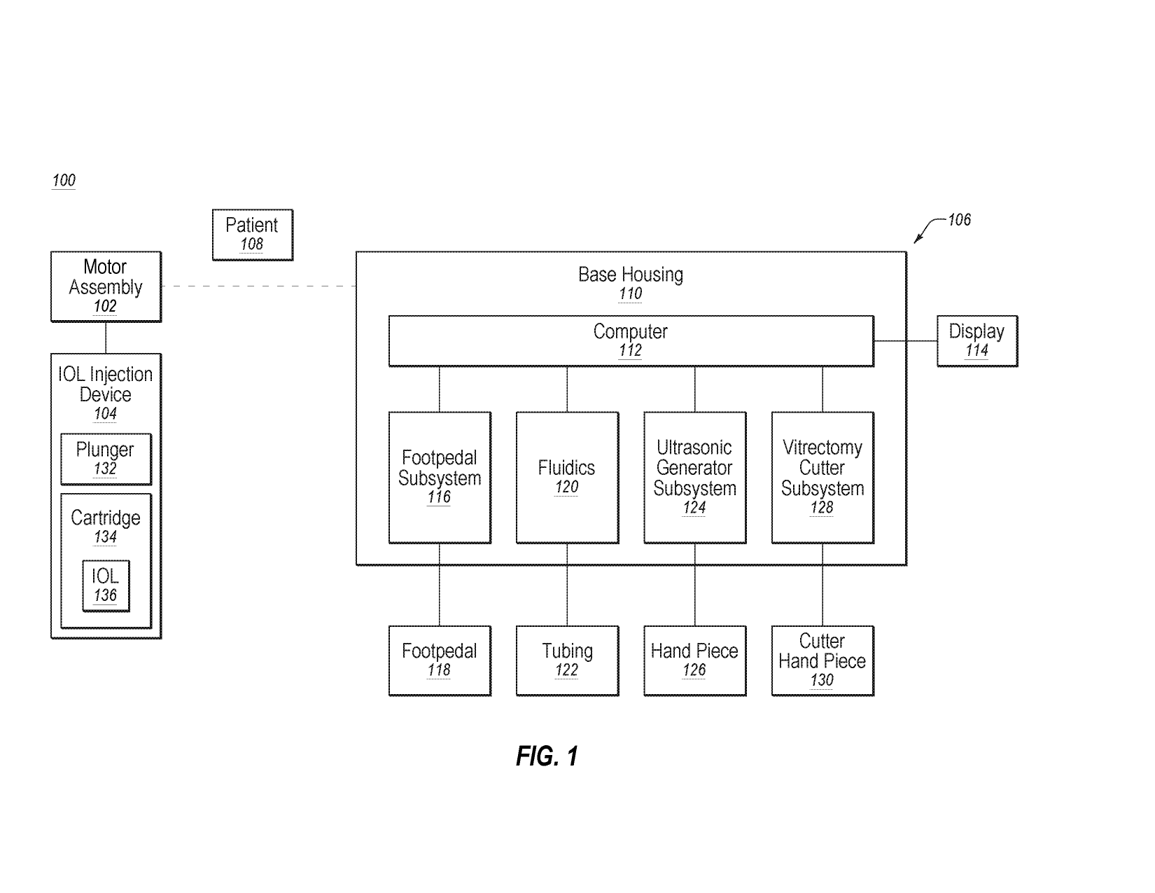

FIG. 1 illustrates an example operating environment 100 in which a motor assembly 102 for an IOL injection device 104 may be implemented, arranged in accordance with at least one embodiment herein. The environment 100 includes the IOL injection device 104 as well as a phacoemulsification surgical console 106 (hereinafter “console 106”) and a patient 108. The console 106 may include a base housing 110 with a computer system 112 disposed therein and an associated display screen 114 showing data relating to system operation and performance during an emulsification surgical procedure. The console 106 may also include a number of subsystems that may be used together to perform an emulsification surgical procedure. For example, the subsystems may include a foot pedal subsystem 116 that includes, for example, a foot pedal 118, a fluidics subsystem 120 that includes an irrigation system and an aspiration system that deliver fluid to and aspirate fluid from the eye through flexible tubing 122, an ultrasonic generator subsystem 124 that includes an ultrasonic oscillation hand piece 126 with a cutting needle, and a pneumatic vitrectomy cutter subsystem 128 that includes a vitrectomy hand piece 130. These subsystems may overlap and cooperate to perform various aspects of the procedure.

The IOL injection device 104 may include a plunger 132 and a cartridge 134 with an IOL 136. In general, the IOL 136 may be loaded into the cartridge 134 (or may come pre-loaded in the cartridge 134), and the cartridge 134 may then be loaded into the IOL injection device 104. With the cartridge 134 and IOL 136 loaded in the IOL injection device 104, the IOL injection device 104 may be operated to axially translate the plunger 132, which in turn deploys the IOL 136 from the cartridge 134, e.g., into the eye of the patient 108. The IOL injection device 104 may be manually operated or may be motorized by the motor assembly 102.

The IOL injection device 104 in some embodiments includes a body, a plunger rod (e.g., the plunger 132 of FIG. 1), and an adjustment knob that experience rotational and/or translational movement relative to one another. In the discussion that follows, it is assumed that the body is the point of reference and movement of the plunger rod and/or the adjustment knob is described with reference and/or relative thereto. For example, the body may be grasped by a surgeon who may manually rotate the adjustment knob, or may operate the motor assembly 102 to rotate the adjustment knob, relative to the body. The adjustment knob may be threadably coupled to the body and may be fixed axially with respect to the plunger rod such that rotation of the adjustment knob causes the adjustment knob, as well as the plunger rod, to translate axially relative to the body.

In general, the motor assembly 102 may include at least two components, including a first component which is angularly fixed with respect to the body and/or the plunger rod of the IOL injection device 104 and a second component which is angularly fixed with respect to the adjustment knob of the IOL injection device 104. The motor assembly 102 is operated to rotate the adjustment knob and thereby axially translate the plunger rod 132 to deploy the IOL 136 from the cartridge 134. The motor assembly 102 may have its own power supply, such as a battery, or it may be coupled to the console 106 which serves as the power supply (e.g., electrical or fluid power supply) or to other external power supply. The connection between the motor assembly 102 and the console 106 is shown in dashed lines to indicate that this connection is optional and may not be present in all embodiments. In some embodiments in which the motor assembly 102 is hydraulically powered, the motor assembly 102 may be hydraulically coupled to the console 106, e.g., through the tubing 122.

Modifications, additions, or omissions may be made to FIG. 1 without departing from the scope of the present disclosure. For example, the environment 100 may include more or fewer elements depending on the implementation. Further, the environment 100 may be configured to perform any number of operations as compared to those explicitly described.

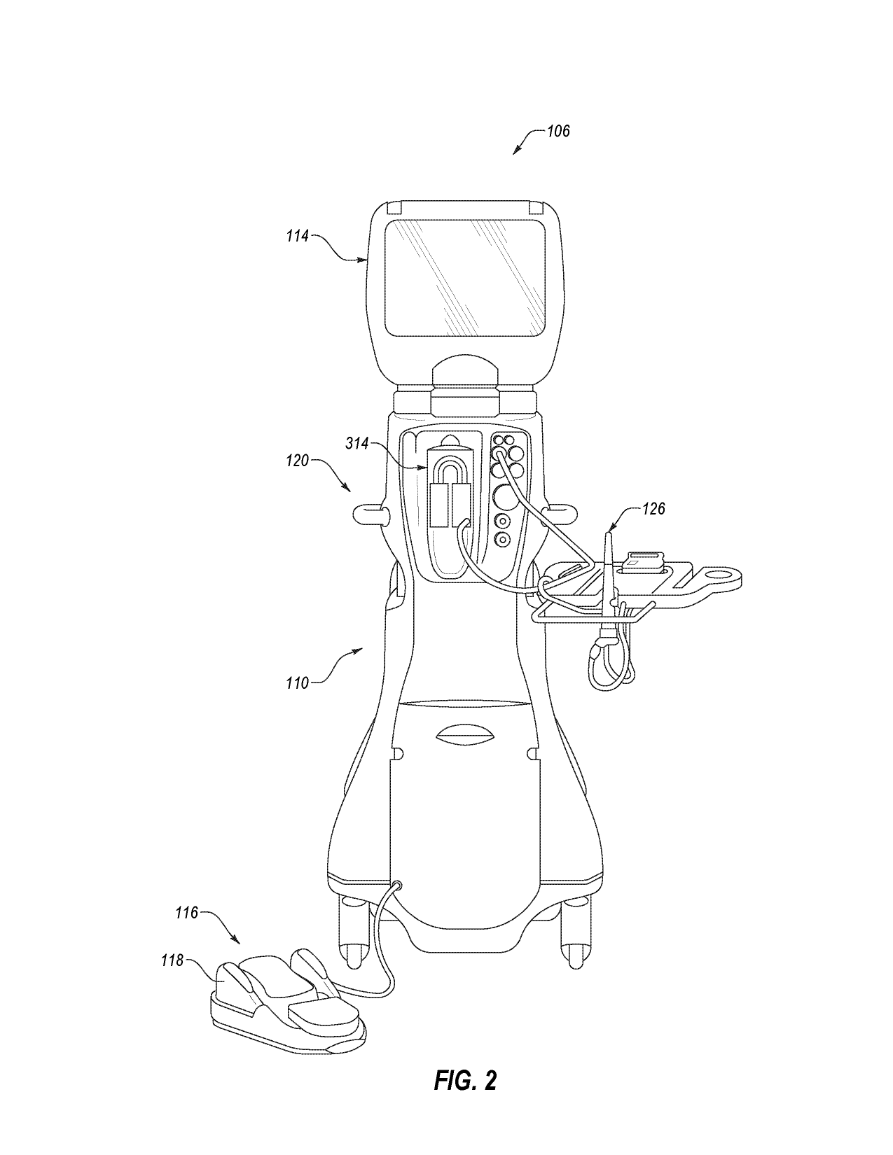

FIG. 2 illustrates an example implementation of the console 106 of FIG. 1, arranged in accordance with at least one embodiment herein. Modifications, additions, or omissions may be made to FIG. 2 without departing from the scope of the present disclosure. For example, the console 106 may include more or fewer elements depending on the implementation. Further, the console 106 may be configured to perform any number of operations as compared to those explicitly described.

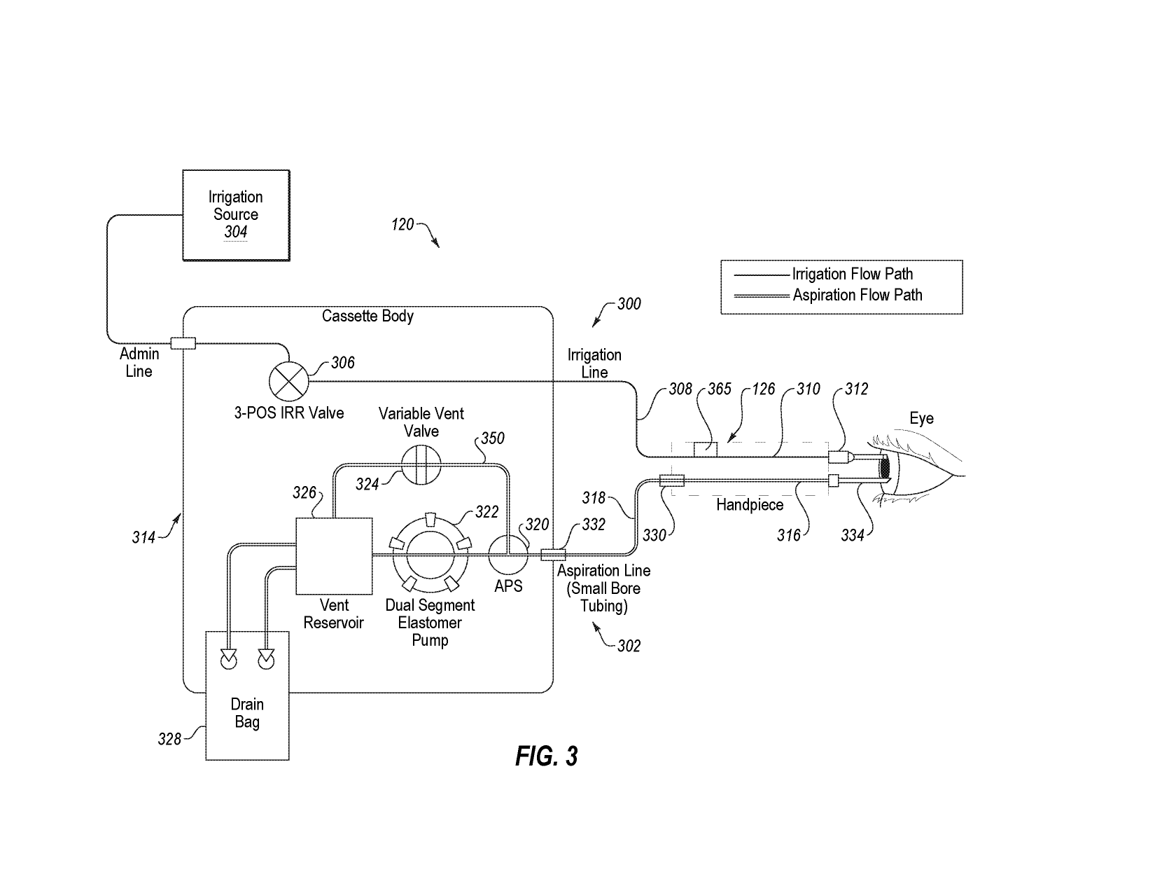

FIG. 3 illustrates a schematic showing the fluidics subsystem 120 and the hand piece 126, arranged in accordance with at least one embodiment herein. The fluidics subsystem 120 includes an irrigation system 300 and an aspiration system 302, each in communication with the hand piece 126. The irrigation system 300 includes an irrigation source 304 as a sterile solution reservoir, an irrigation valve 306 that regulates flow from the reservoir to the surgical site, a flexible irrigation tubing 308, an irrigation path 310 in the hand piece 126, and a sleeve 312 that may be considered a component of the hand piece 126. The irrigation source 304 may be a mechanically pressurized fluid source such as, for example, a clamping pressure system. In another embodiment, the irrigation source 304 may be a source suspended by a pole (e.g., an IV pole) which may or may not be adjustable. Other fluid sources also are contemplated.

The irrigation system 300 extends between the sterile solution reservoir 304 and the hand piece 126, and carries fluid to the surgical site (labeled in FIG. 3 as an eye, e.g., of the patient 108 in FIG. 1). In one example, the sterile fluid is a balanced salt solution (BSS), however, other fluids may be used. The flexible irrigation tubing 308 may be formed in part of the flexible tubing 122 in FIG. 1. In some embodiments, the irrigation tubing 308 is formed of multiple segments, with some segments being rigid and others being flexible. Also, in some embodiments, at least a portion of the irrigation system 300 is formed in a cassette 314 that cooperates with the console 106 in FIG. 1 to provide fluid communication between the sterile solution reservoir 304 and the patient's eye. As indicated above, in some embodiments, the irrigation sleeve 312 is disposed about the cutting needle to provide irrigating fluid flow to the eye during the surgical procedure.

The aspiration system 302 includes an aspiration path 316 in the hand piece 126, a hand piece pressure sensor (HPS) 365 located within the hand piece 126, a small bore flexible aspiration tubing 318, an aspiration pressure sensor (APS) 320, a pump 322, a vent valve 324, a drain line reservoir 326, and a drain reservoir 328. In an embodiment, the HPS 365 may be disposed at the irrigation path of the hand piece 126. In another embodiment, the HPS 365 may be disposed at the aspiration path of the hand piece 126. A hand piece connector 330 connects the aspiration path 316 in the hand piece 126 to the small bore flexible aspiration tubing 318. A cassette connector 332 connects the flexible aspiration tubing 318 to the cassette aspiration line in the cassette 314. As can be seen, the aspiration system 302 extends from the surgical site (eye) to the drain reservoir 328. It carries away fluid used to flush the eye as well as any emulsified particles. As described above with reference to the flexible irrigation tubing 308, at least a portion of the small bore flexible aspiration tubing 318 may be formed of the flexible tubing 122. In some embodiments, the aspiration system 302 is formed of multiple segments, with some segments being rigid and others being flexible. Also, in some embodiments, at least a portion of the aspiration system 302 is formed in the cassette 314 that cooperates with the console 106 in FIG. 1 to provide fluid communication between the hand piece 126 and the drain reservoir 328. In some embodiments, the drain reservoir 328 may in fact be a drain instead of a self-contained reservoir. As indicated above, in some embodiments, the aspiration system 302, including the aspiration fluid path 316, is in fluid communication with the bore of the cutting tip (labeled 334 in FIG. 3) of the hand piece 126 and is used to aspirate fluid and emulsified particles through the needle bore and into the aspiration system 302 during the surgical procedure.

When vacuum is generated within the aspiration pathway due to a flow obstruction, such as when lens fragments enter and clog portions of the aspiration pathway during a surgery, the console 106 may detect the vacuum, or pressure difference, via pressure sensor 320 installed at the pump 322 and/or pressure sensor 365 installed in the hand piece 126. The console 106 may control the vent valve 324 to open to relieve the vacuum in the aspiration pathway and to reduce the effect of occlusion break. This may reduce the magnitude of resulting surge and maintain a predetermined level of vacuum so as not to lessen the efficiency of lens removal.

The same, similar, or different cassette 314 may be used to couple the motor assembly 102 of FIG. 1 to the fluidics subsystem 120 of the console 106 of FIG. 1 in embodiments in which the motor assembly 102 is a hydraulic motor assembly. In these and other embodiments, the cassette 314 may include an output line to the motor assembly 102 (which output line of the cassette 314 may be coupled to an input of the hydraulic motor assembly 102) as well as an input line from the motor assembly 102 (which input line of the cassette 314 may be coupled to an output of the hydraulic motor assembly 102) as well as a pump or other means to force fluid through, sequentially, the output line, the hydraulic motor assembly 102, and the input line.

Modifications, additions, or omissions may be made to FIG. 3 without departing from the scope of the present disclosure. For example, the fluidics subsystem 120 and/or the hand piece 1206 may include more or fewer elements depending on the implementation. Further, the fluidics subsystem 120 and/or the hand piece 126 may be configured to perform any number of operations as compared to those explicitly described.

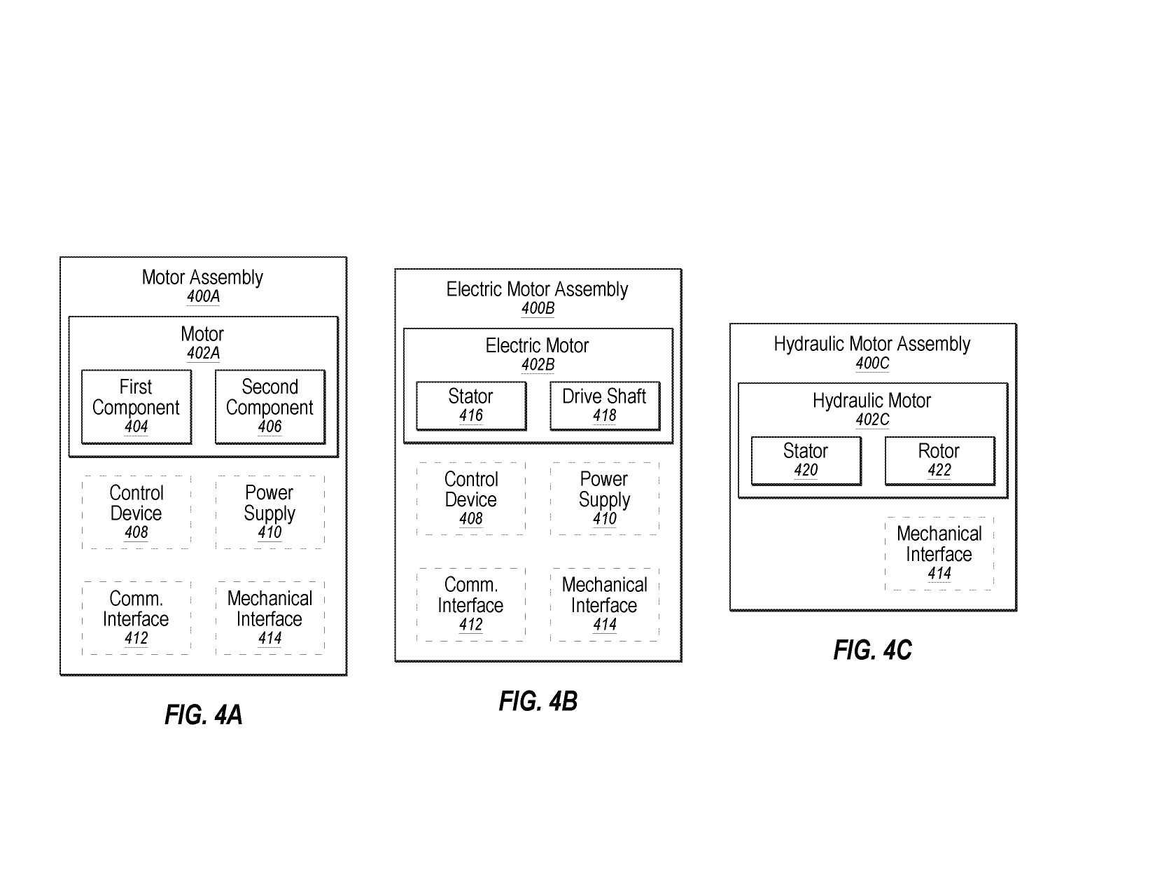

FIGS. 4A-4C illustrate various example motor assemblies 400A, 400B, 400C that may be implemented in the environment 100 of FIG. 1, arranged in accordance with at least one embodiment herein.

The motor assembly 400 of FIG. 4A includes a motor 402A that includes a first component 404 and a second component 406. When used with an IOL injection device, the first component 404 may be angularly fixed with respect to a first portion of the IOL injection device, such as the body or plunger rod of the IOL injection device, while the second component 406 may be angularly fixed with respect to a second portion of the IOL injection device, such as the adjustment knob of the IOL injection device. The first component 404 may include a stator, or a drive shaft or rotor of the motor assembly 400A. The second component 406 may include the other of the drive shaft or rotor, or the stator of the motor assembly 400A.

The motor assembly 400A may optionally further include one or more of a control device 408, a power supply 410, a communication interface 412, and a mechanical interface 414.

The control device 408, whether included as part of the motor assembly 400A or provided externally from the motor assembly 400A, may be configured to operate the motor 402A, e.g., to turn the motor on or off so that the first or second component 404, 406 rotates relative to the other, and/or to increase or decrease a rotational speed of the first or second component 404, 406. In some embodiments, the control device 408, when included as part of the motor assembly 400A, may include a potentiometer, a slide button, and/or other components or structure to operate the motor 402A.

The power supply 410, whether included as part of the motor assembly 400A or provided externally from the motor assembly 400A, may be configured to provide power to the motor 402A. The power supply 410 may generally include a battery, an electrical power supply from a phacoemulsification surgical console or other source, a fluid power supply from a phacoemulsification surgical console or other source, or other power supply. In embodiments in which the power supply 410 is included as part of the motor assembly 400A, the power supply 410 may include a battery.

The communication interface 412, whether or not included as part of the motor assembly 400A, may be configured to wirelessly and/or communicatively couple the motor 402A to another component, such as a control device 408. In such embodiments, the control device may include or be coupled to another communication interface, and the communication interface 412 of the motor assembly 400A may be communicatively coupled to the communication interface of the control device 408 to permit the motor 402A to be wirelessly controlled. As a particular example, a foot pedal of a phacoemulsification surgical console may include or be coupled to a communication interface that may be communicatively coupled to the communication interface 412 of the motor assembly 400A to allow the motor 402A to be wireless controlled by the foot pedal through the communication 412 of the motor assembly 400A and the communication interface of the foot pedal. In these and other embodiments, the communication interface 412 and/or other communication interfaces herein may include a Bluetooth chip, a Wi-Fi (e.g., 802.11) chip, and/or other wireless communication chips or devices.

The mechanical interface 414, whether or not included as part of the motor assembly 400A, may be configured to mechanically couple and/or interface the first component 404 of the motor 402A to or with the first portion of the IOL injection device and/or the second component 406 of the motor 402A to or with the second portion of the IOL injection device. As an example, the mechanical interface 414 may include a housing or other structure that has one end coupled to a body of an IOL injection device and another end coupled to and supporting the first component 404 (e.g., a stator) of the motor 402A. As another example, the mechanical interface 414 may include a turbine or a flex shaft cable coupled between the second component 406 (e.g., a drive shaft) of the motor 402A and an adjustment knob of the IOL injection device. As still another example, the mechanical interface 414 may include a clamp to secure or attach the first or second component 404, 406 of the motor 402A to a corresponding portion of the IOL injection device.

FIGS. 4B and 4C include example implementations of the motor assembly 400A of FIG. 4A as, respectively, an electric motor assembly 400B and a hydraulic motor assembly 400C. The electric motor assembly 400B includes an electric motor 402B having a stator 416 and a drive shaft 418. The drive shaft 418 may be included as part of or coupled to a rotor of the electric motor 402B. The stator 416 may be included in or correspond to the first component 404 or the second component 406 while the drive shaft 418 may be included in or correspond to the other of the second component 406 or the first component 404. The hydraulic motor assembly 400C includes a hydraulic motor 402C having a stator 420 and a rotor 422. One or both of the electric motor assembly 400B and/or the hydraulic motor assembly 400C may include one or more other components than depicted in FIGS. 4B-4C, such as seals (e.g., o-rings) for the hydraulic motor assembly 400C, and/or other components.

Electric motors and hydraulic motors typically include a stator and a rotor to respectively designate a first component or set of components of the motor that typically remains fixed and a second component or set of components of the motor that typically rotates relative to the first component. Notwithstanding the foregoing, the rotor may instead be fixed, in which case operation of the electric motor may cause the stator to rotate relative to the rotor while the rotor remains fixed. The terms “stator” and “rotor” are used herein to refer to the components of an electric or hydraulic motor typically referred to as a “stator” or “rotor”, even though in some embodiments the part referred to as the “rotor” may be fixed while the part referred to as the “stator” may rotate relative to the rotor.

The electric motor assembly 400B of FIG. 4B may optionally further include one or more of the control device 408, the power supply 410, the communication interface 412, and/or the mechanical interface 414. Alternatively or additionally, the hydraulic motor assembly 400C of FIG. 4C may optionally further include the mechanical interface 414.

Modifications, additions, or omissions may be made to FIGS. 4A-4C without departing from the scope of the present disclosure. For example, the motor assemblies 400A, 400B, 400C may include more or fewer elements depending on the implementation. Further, the motor assemblies 400A, 400B, 400C may be configured to perform any number of operations as compared to those explicitly described.

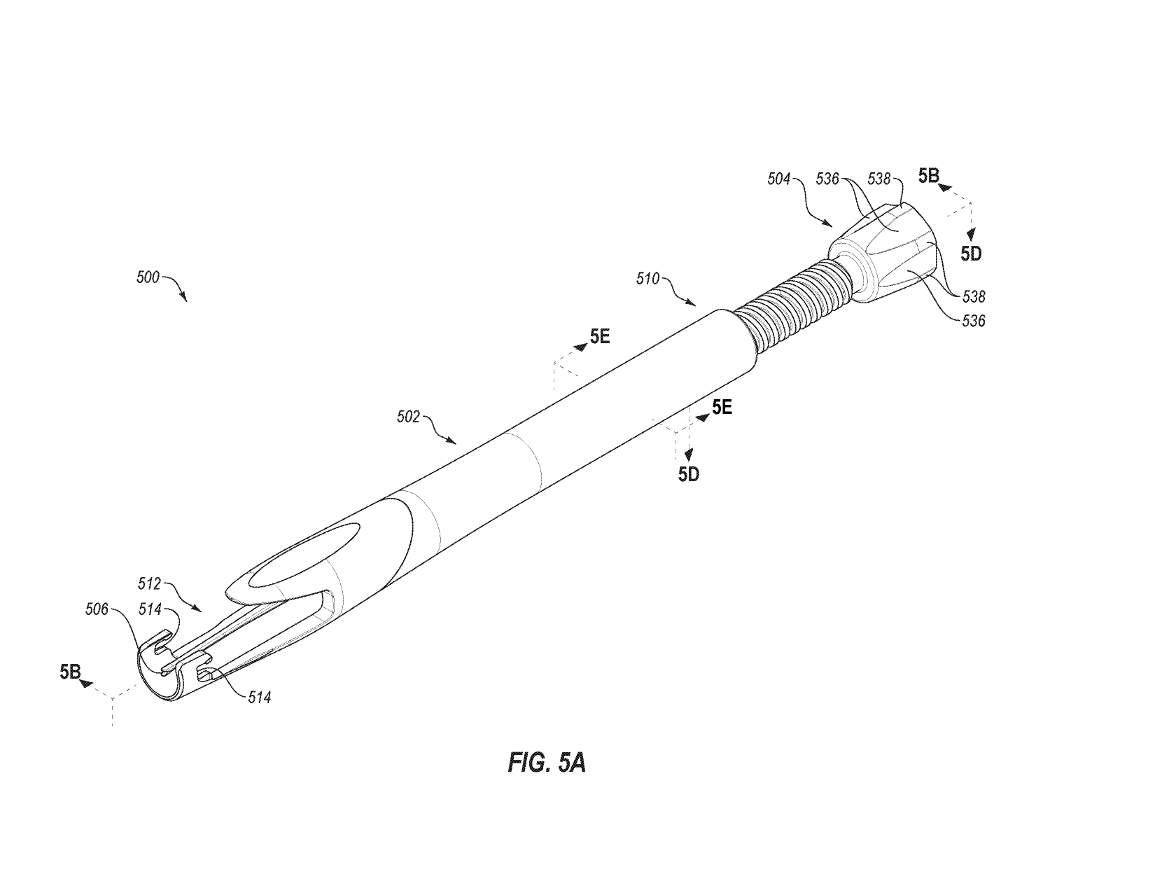

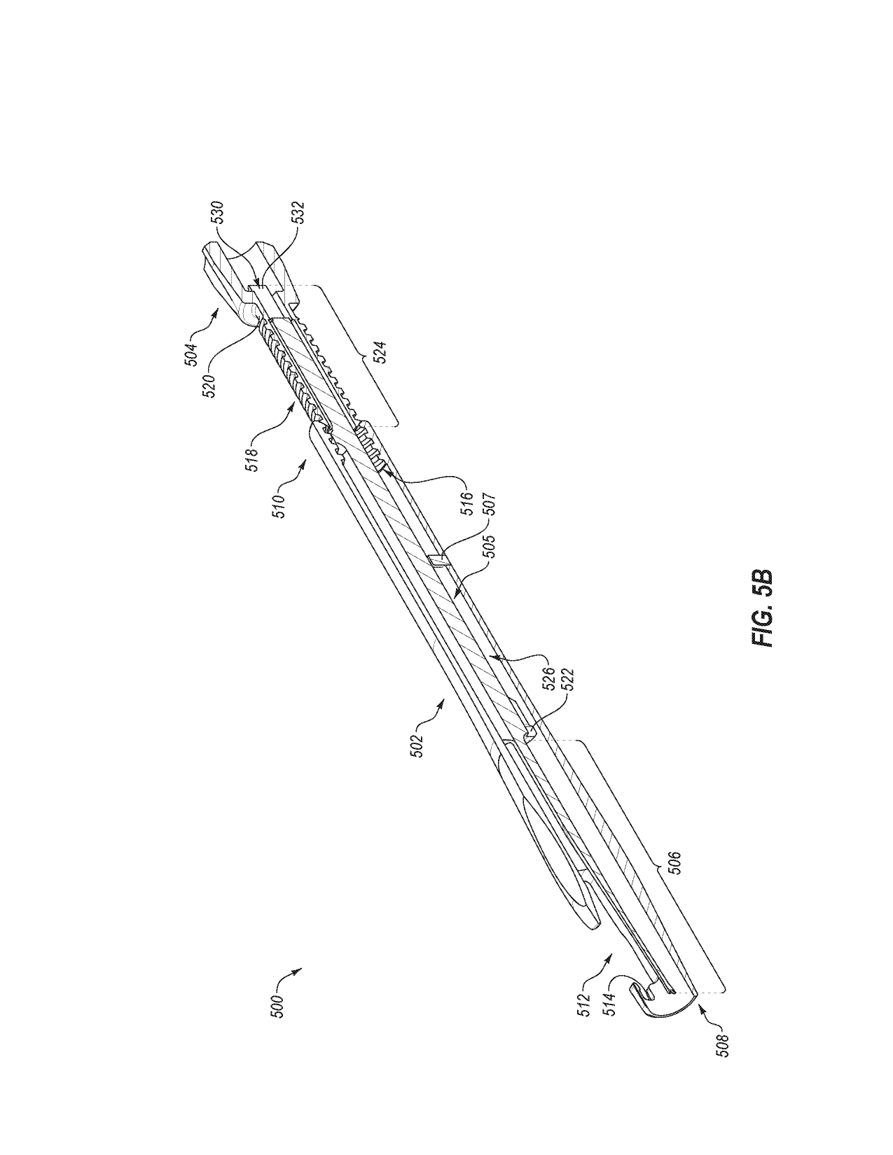

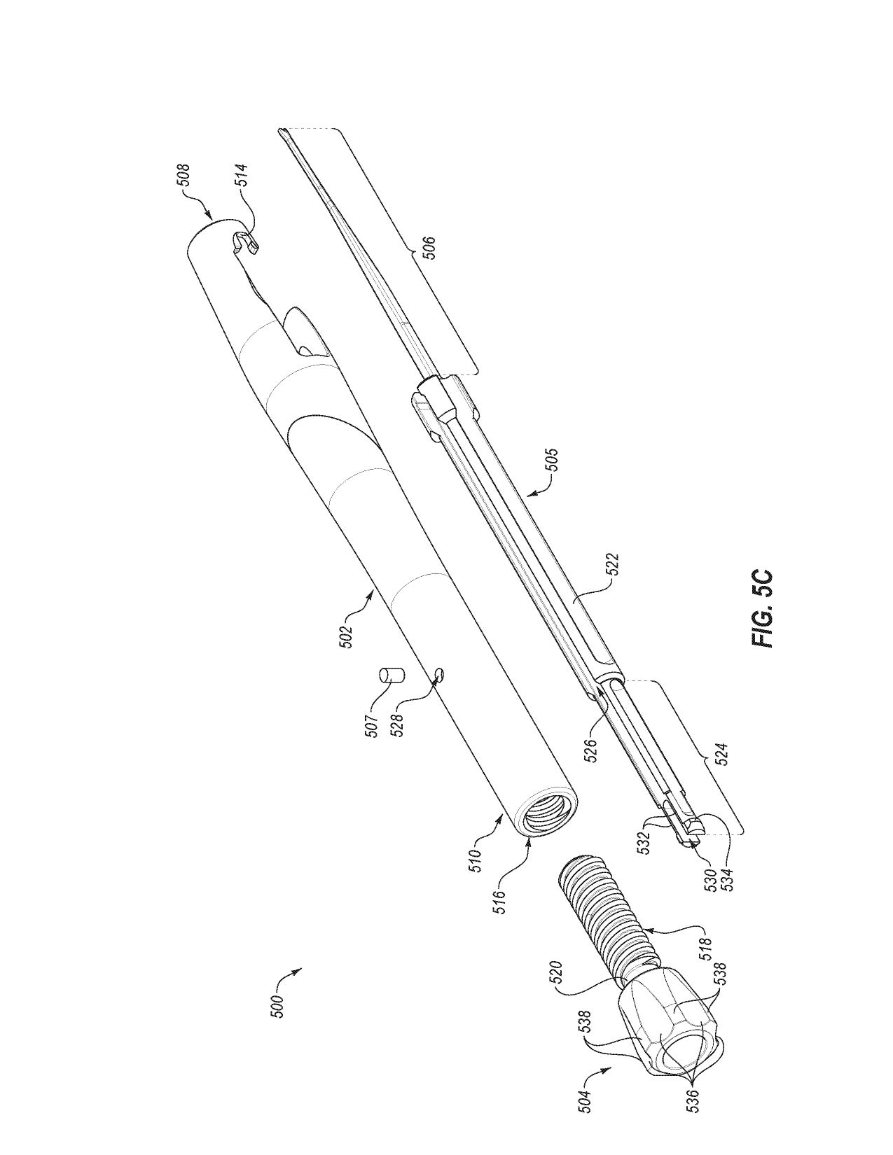

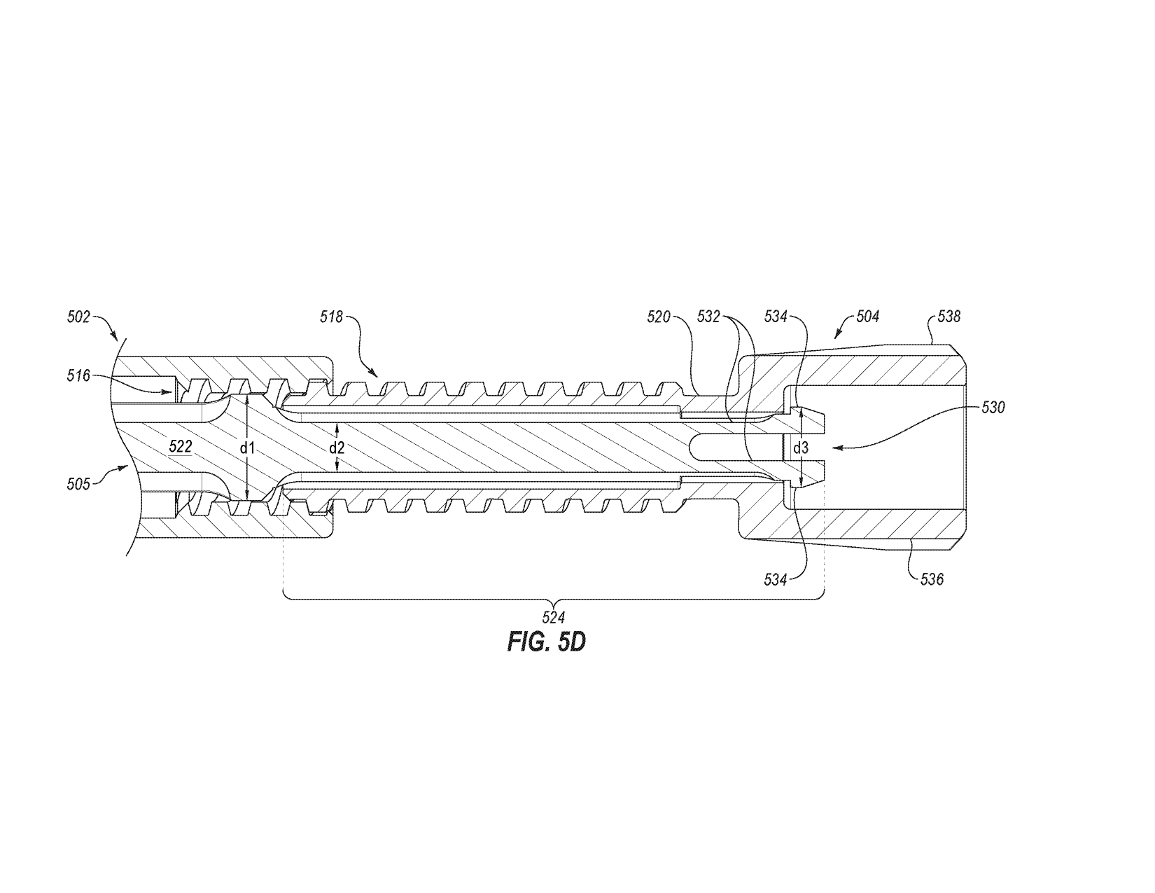

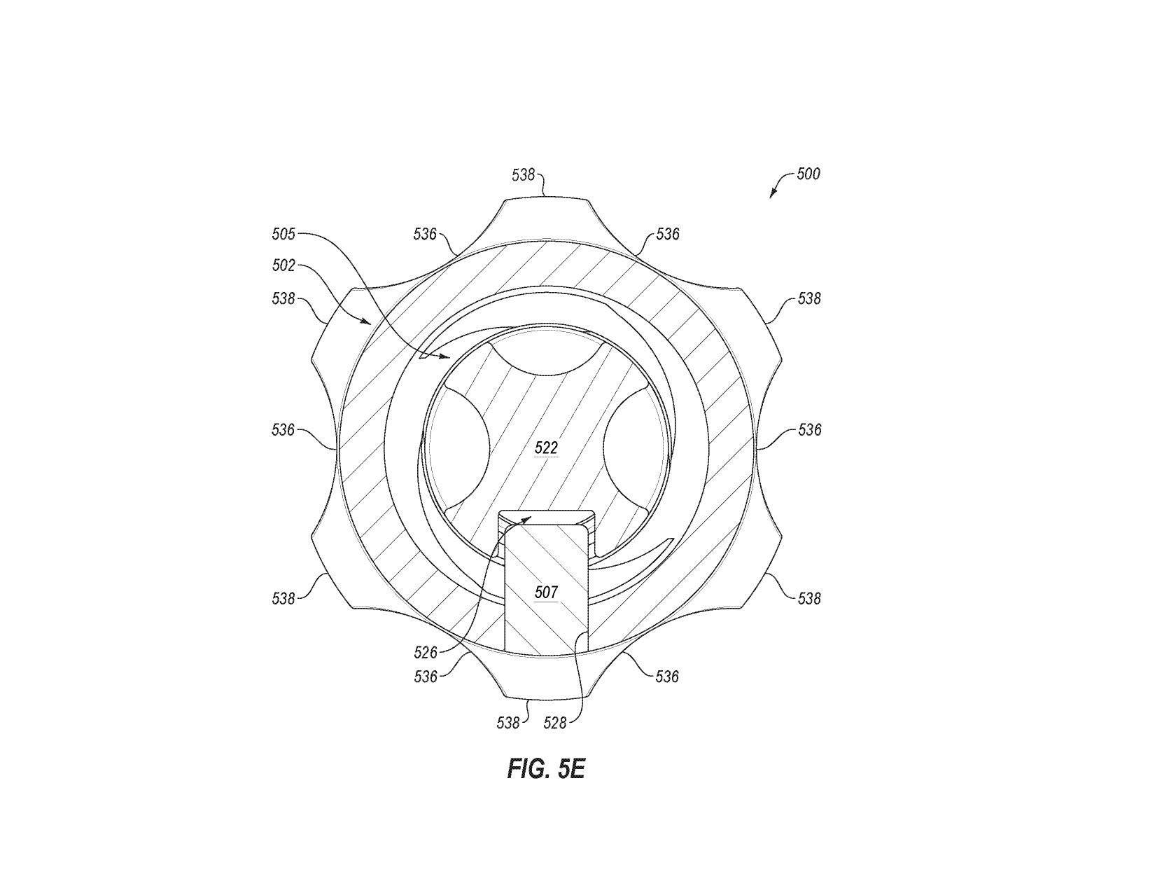

FIGS. 5A-5E illustrate an example IOL injection device 500 to which a motor assembly may be attached, arranged in accordance with at least one embodiment described herein. The IOL injection device 500 may include, be included in, or correspond to the IOL injection device 104 of FIG. 1 or other IOL injection devices described herein. The motor assembly attached to the IOL injection device 500 may include any of the motor assemblies described herein, including the motor assemblies 400A, 400B, 400C of FIGS. 4A-4C.

FIG. 5A is a front perspective view of the IOL injection device 500. FIG. 5B is a front perspective cross-sectional view of the IOL injection device 500 taken along cutting plane 5B-5B in FIG. 5A. FIG. 5C is an exploded upside-down rear perspective view of the IOL injection device 500. FIG. 5D is an overhead cross-sectional view of a portion of the IOL injection device 500 taken along cutting plane 5D-5D in FIG. 5A. FIG. 5E is a front cross-sectional view of the IOL injection device 500 taken along cutting plane 5E-5E in FIG. 5A.

As illustrated, the IOL injection device 500 may include a body 502 or housing, an adjustment knob 504, a plunger rod 505 that includes a plunger tip 506, and an alignment post 507 (FIGS. 5C., 5E). The body 502 includes a front or distal end 508 and a rear or proximal end 510.

The front end 508 defines a cartridge receptacle 512 to receive a cartridge loaded with an IOL. The cartridge receptacle 512 includes slots 514.

The rear end 510 defines internal threads 516 that threadably engage external threads 518 on a shaft 520 of the adjustment knob 504. In consideration of the threadable engagement between the body 502 and the adjustment knob 504, rotating the adjustment knob 504 relative to the body 502 in one direction (e.g., clockwise in this example) causes the adjustment knob 504 to advance or translate axially into or towards the body 502. On the other hand, rotating the adjustment knob 504 relative to the body 502 in the other direction (e.g., counterclockwise in this example) causes the adjustment knob 504 to retreat or translate axially away from the body 502.

The plunger rod 505 includes a plunger body 522, the plunger tip 506 extending from a front of the plunger body 522, and a rod end 524 extending from a rear of the plunger body 522. The plunger body 522 defines an alignment channel 526 along its underside or ventral side, as best depicted in FIGS. 5C and 5E. In the assembled IOL injection device 500, the alignment post 507 is secured in a hole 528 (FIG. 5C) defined in an underside or ventral side of the body 502 and may be retained within the hole 528 via a friction fit, adhesive, or other means. As depicted in FIG. 5E, the alignment post 507 extends into the alignment channel 526 of the plunger body 522. With the alignment post 507 secured in the hole 528 of the body 502 and extending into the alignment channel 526 of the plunger body 522, the plunger rod 505 may be unable to rotate relative to the body 502 (beyond minor relative rotations that may arise from, e.g., the alignment channel 526 being slightly wider than a diameter of the alignment post 507). However, the alignment channel 526 extends axially along the plunger body 522, i.e., in a direction parallel to a longitudinal axis of the plunger rod 505, such that the plunger rod 505 may translate axially relative to the body 502 and the alignment post 507. Thus, the plunger rod 505 may be rotationally or angularly fixed with respect to the body 502 while being translatable axially relative to the body 502.

The adjustment knob 504 may engage the plunger rod 505 to translate the plunger rod 505 axially forward (i.e., in a direction from the rear end 510 to the front end 508 of the body 502) or backward (i.e., in a direction from the front end 508 to the rear end 510 of the body 502) relative to the body 502. Referring to FIG. 5D, in this and other embodiments, a front of the shaft 520 of the adjustment knob 504 may engage the plunger body 522 to translate the plunger rod 505 axially forward while a rear of the shaft 520 of the adjustment knob 504 may engage a rear of the rod end 524 to translate the plunger rod 505 axially backward.

In more detail, in this example, the rear end of the plunger body 522 may have a first diameter d1 that is larger than an inner diameter of the shaft 520 of the adjustment knob 504 while the rod end 524 may have a second diameter d2 that is smaller than the inner diameter of the shaft 520. The diameter d2 of the rod end 524 may transition to the diameter d1 of the rear end of the plunger body 522 gradually (as shown) or abruptly. The smaller diameter d2 of the rod end 524 permits the rod end 524 to be inserted into the shaft 520 of the adjustment knob 504 up until the shaft 520 reaches the plunger body 522 having the larger diameter. Because the plunger body 522 has a larger diameter than the inner diameter of the shaft 520 of the adjustment knob 504, any forward axial translation of the adjustment knob 504 will cause forward axial translation of the plunger rod 505.

In further detail, and with continued reference to FIG. 5D, a rear end of the rod end 524 may define a slot 530 between retention posts 532 that may be resiliently deformable. For example, the retention posts 532 may flex inward or outward under external force and return to an unflexed state (as depicted in FIG. 5D) in the absence of the external force. Each post 532 has a protrusion 534 with the plunger end 524 having a third diameter d3 through the protrusions 534. The third diameter d3 through the protrusions 534 may be larger than the inner diameter of the shaft 520 of the adjustment knob 504 when the posts 532 are in an unflexed state. When the adjustment knob 504 is installed on the rod end 524 of the plunger rod 505, the retention posts 523 may be pinched together as they pass through the shaft 520 of the adjustment knob 504 and, after the protrusions 534 clear the shaft 520 and are no longer constrained by it, the posts 532 may return to (or at least towards) the unflexed state. Because the third diameter d3 through the protrusions 534 of the posts 532 is larger than the inner diameter of the shaft 520 of the adjustment knob 504, any backward axial translation of the adjustment knob 504 will cause backward axial translation of the plunger rod 505.

Accordingly, FIGS. 5A-5E illustrate how the plunger rod 505 may be rotationally fixed with respect to the body 502 and axially fixed with respect to the adjustment knob 504. As such, when the adjustment knob 504 rotates and translates axially relative to the body 502, the plunger rod 505 does not rotate relative to the body 502 but it does translate axially relative to the body 502.

In some embodiments, one or more of the body 502, the adjustment knob 504, and/or the plunger rod 505 of the IOL injection device 500 may have one or more engagement features that may be engaged by a motor assembly to couple the motor assembly to the IOL injection device 500. For example, the slot 530 in the rod end 524 of the plunger rod 505 may receive therein a shaft end of or coupled to a drive shaft or rotor of a motor assembly. As another example, and referring to, e.g., FIGS. 5A, 5C, and 5E, the adjustment knob 504 may include two or more grooves 536 interposed between and/or defined by two or more ridges 538. The grooves 536 and/or ridges 538 may each extend axially. In some embodiments, the motor assembly may engage with the adjustment knob 504, and particularly with the grooves 536 and/or the ridges 538, similar to the way two intermeshed gears engage each other, so that rotation of a portion of the motor assembly coupled to the adjustment knob 504 also causes the adjustment knob 504 to rotate.

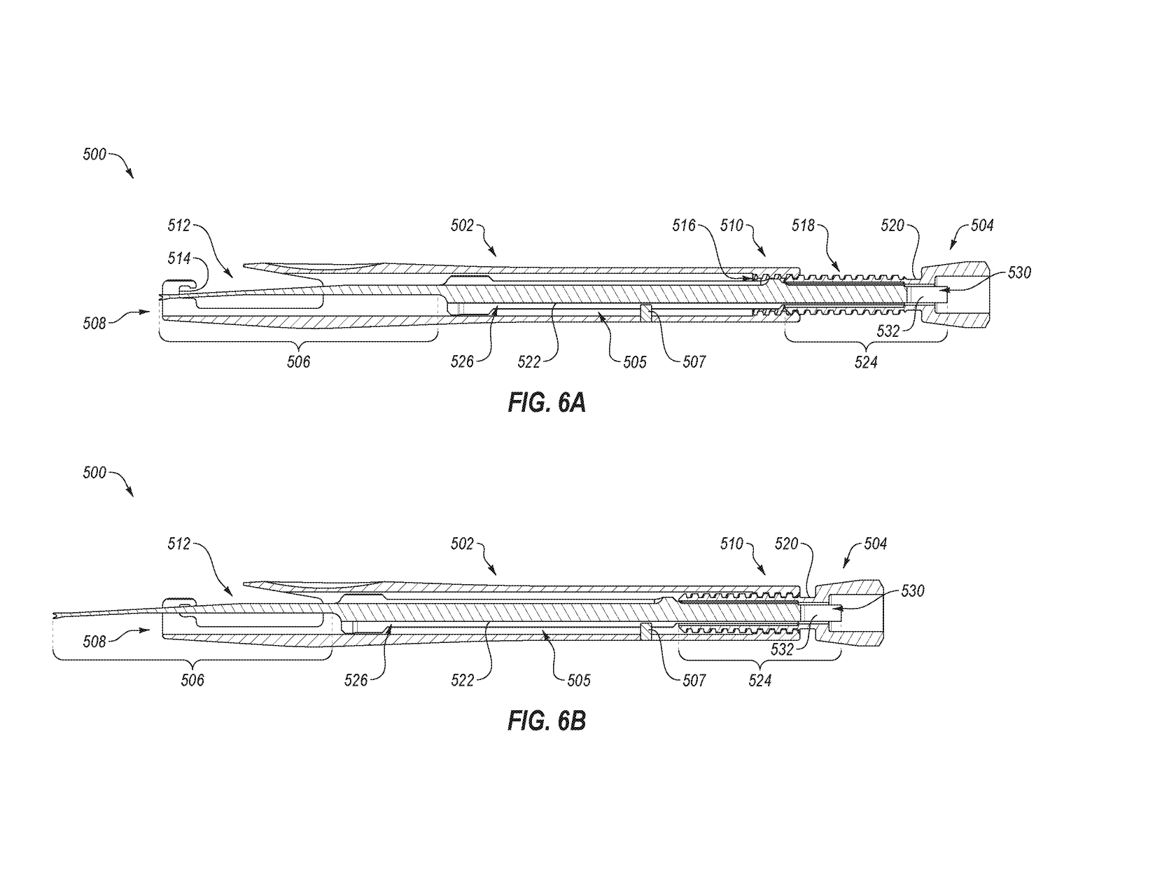

FIGS. 6A and 6B are cross-sectional views of the IOL injection device 500 of FIGS. 5A-5E with the adjustment knob 504 and the plunger rod 505 at two different axial locations relative to the body 502, arranged in accordance with at least one embodiment described herein. Each of the cross-sectional views of FIGS. 6A and 6B is taken along the cutting plane 5B-5B of FIG. 5A. FIG. 6A shows the adjustment knob 504 barely screwed into the body 502 with the forward most end of the plunger tip 506 almost at the same axial location as the forward most end of the front end 508 of the body 502. FIG. 6B shows the adjustment knob 504 almost entirely screwed into the body 502 with the forward most end of the plunger tip 506 well past the forward most end of the front end 508 of the body 502 in the axial direction. The adjustment knob 504 and the plunger rod 505 may be translated relative to the body 502 between the two positions depicted in FIGS. 6A and 6B (or even beyond these positions in some embodiments) by rotating the adjustment knob 504 relative to the body 502.

Modifications, additions, or omissions may be made to FIGS. 5A-6B without departing from the scope of the present disclosure. For example, the IOL injection device 500 of FIGS. 5A-6B may include more or fewer elements depending on the implementation. Further, the IOL injection device 500 may be configured to perform any number of operations as compared to those explicitly described. Additionally, the IOL injection device 500 is provided as one example physical implementation of such a device, but it will be appreciated that the principles of the present disclosure are also applicable to other injection devices.

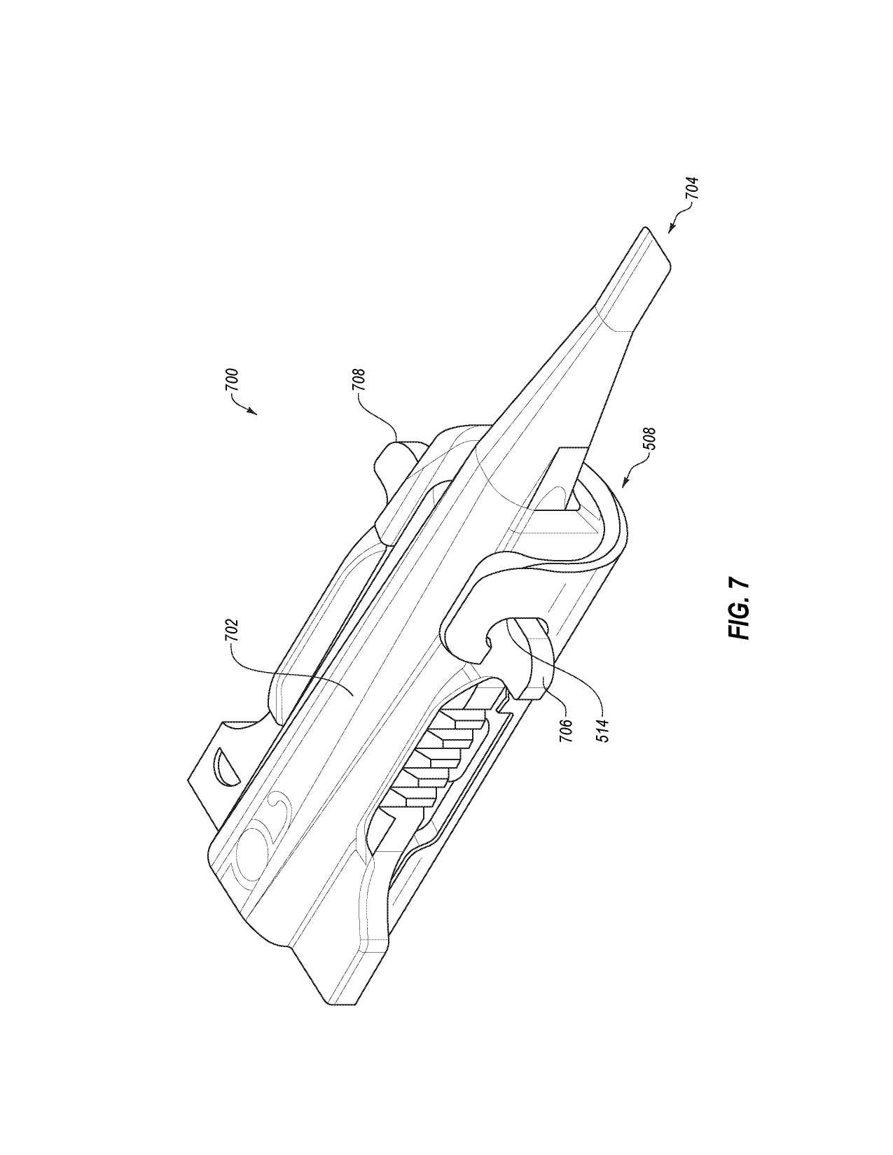

FIG. 7 is a front perspective view of an example cartridge 700 loaded in the IOL injection device 500 of FIGS. 5A-5E, arranged in accordance with at least one embodiment described herein. The cartridge 700 may include, be included in, or correspond to the cartridge 134 of FIG. 1 or other cartridges described herein.

As illustrated, the cartridge 700 includes a body 702 that narrows to a front or distal end 704. Tabs or wings 706, 708 extend from opposing sides of the body 702 and may be received in the slots 514 when the cartridge 700 is installed in the cartridge receptacle 512 of the IOL injection device 500 to secure the cartridge 700 within the cartridge receptacle 512.

In use, an IOL may be loaded into the cartridge 700 through a rear or proximal end (opposite from the front end 704) and the cartridge 700 and IOL may then be loaded into the cartridge receptacle 512 of the IOL injection device 500. A surgeon then inserts the front or distal end 704 of the cartridge 700 into the patient’s eye and injects the IOL into the patient’s eye. In particular, the surgeon operates the IOL injection device 500 to rotate the adjustment knob 504 (either manually or using a motor assembly as described herein) and thereby axially advances the plunger rod 505 relative to the body 502 of the IOL injection device 500. The plunger tip 506 engages a rear of the IOL within the cartridge 700 and as the plunger rod 505 is axially advanced relative to the body 502 of the IOL injection device 500, the plunger tip 506 pushes against and advances the IOL through the cartridge 700 and the IOL exits through the front end 704 of the cartridge 700 into the patient’s eye. The front end 704 of the cartridge 700 may then be removed from the patient’s eye.

Modifications, additions, or omissions may be made to FIG. 7 without departing from the scope of the present disclosure. For example, the cartridge 700 may include more or fewer elements depending on the implementation. Further, the cartridge 700 may be configured to perform any number of operations as compared to those explicitly described.

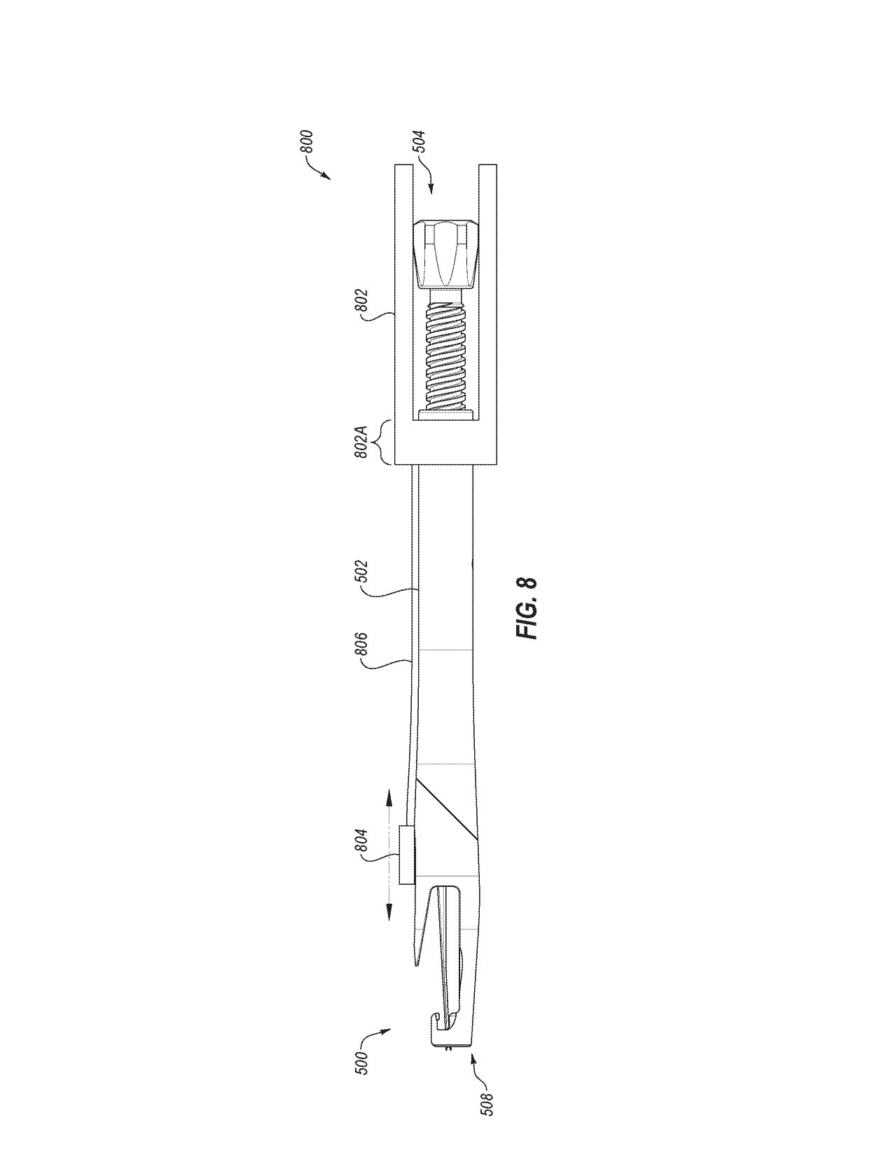

FIG. 8 is a side view of the IOL injection device 500 of FIGS. 5A-5C with an example motor assembly 800, arranged in accordance with at least one embodiment described herein. The motor assembly 800 may include, be included in, or correspond to any of the other motor assemblies described herein.

As illustrated, the motor assembly 800 includes a motor 802 and a control device 804. In general, the motor 802 may include a first component (e.g., a stator, or rotor or drive shaft) removably coupled to the body 502 or the plunger rod 505 and a second component (e.g., the other of the rotor or drive shaft, or stator) removably coupled to the adjustment knob 504.

The control device 804 in FIG. 8 may be or include a potentiometer with a button including or coupled to a sliding contact of the potentiometer, an encoder, or other suitable control device or arrangement. The control device 804 may be coupled to the motor 802 via a wireless or hardwired connection 806. In some embodiments, sliding the button, and particularly the sliding contact, of the control device 804 forward or backward may increase or decrease a voltage supplied through the potentiometer of the control device 804 to the motor 802. Increasing or decreasing the voltage supplied to the motor 802 may increase or decrease the rotational speed of the motor 802 to increase or decrease the axial translation speed of the plunger rod 505 relative to the body 502. The control device 804 of FIG. 8 in the form of a potentiometer and/or button mounted on the body 502 may be used in connection with any of the electric motor assemblies and/or other motor assemblies described herein.

Modifications, additions, or omissions may be made to FIG. 8 without departing from the scope of the present disclosure. For example, the motor assembly 800 may include more or fewer elements depending on the implementation. Further, the motor assembly 800 may be configured to perform any number of operations as compared to those explicitly described.

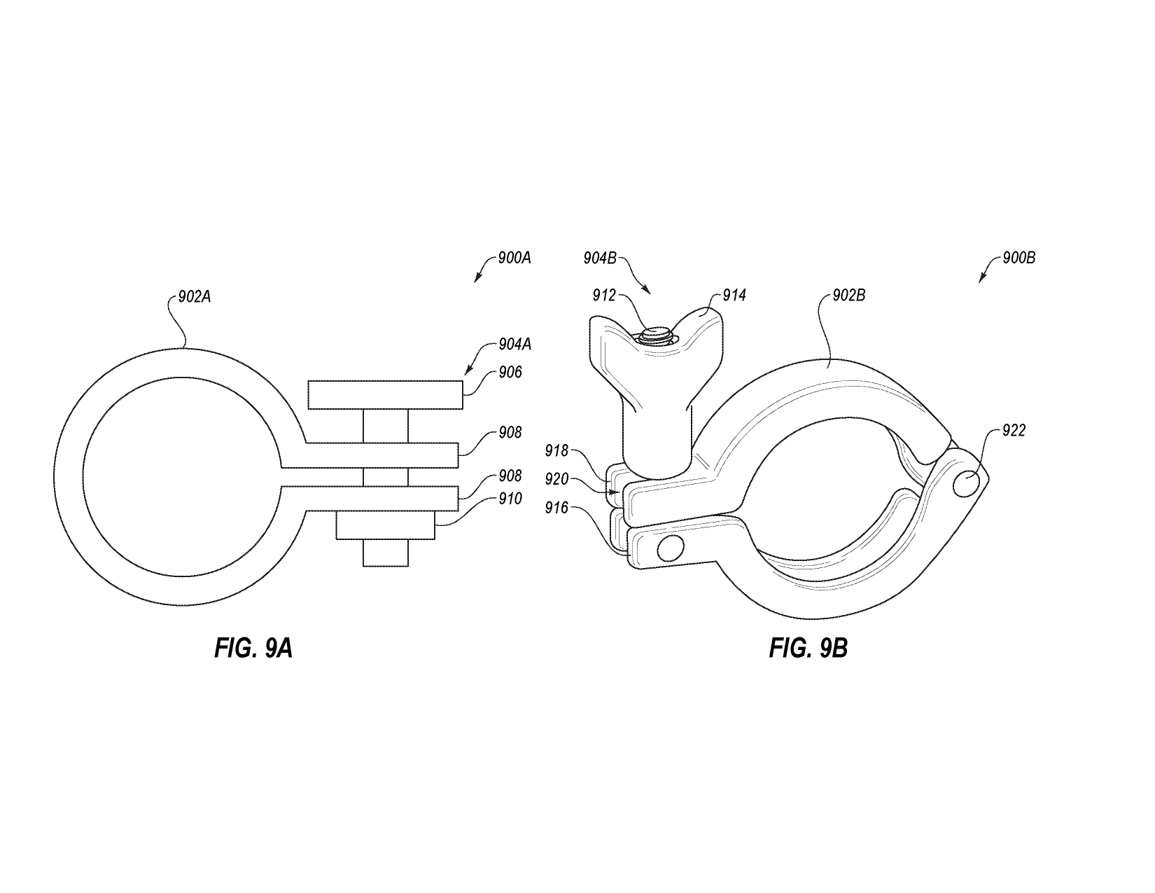

FIGS. 9A-9B illustrate example clamps 900A, 900B to couple a motor assembly to an IOL injection device, arranged in accordance with at least one embodiment described herein. The clamps 900A, 900B may be used to, e.g., clamp an end 802A of the motor 802 of FIG. 8 to the body 502 of the IOL injection device 500. Although described in the context of FIG. 8, the clamps 900A, 900B of FIGS. 9A-9B or any other clamp may be used to couple any desired motor described to any desired IOL injection device.

Each of the clamps 900A, 900B may generally include a band 902A, 902B and a tightener 904A, 904B. Each band 902A, 902B may have a shape that generally corresponds to or may conform to a cross-sectional shape of the part or parts being clamped (e.g., the body 502 of the IOL injection device 500). In this example, each band 902A, 902B is generally circular and corresponds to a generally circular cross-sectional shape of the end 802A of the motor 802. The shape of the band 902A, 902B may be expandable and/or shrinkable through operation of the tightener 904A, 904B. For example, when the shape of a given band 902A, 902B is larger than a cross-sectional shape of the end 802A of the motor 802 as mounted on the body 502 of the IOL injection device, the band 902A, 902B may be maneuvered into position at the end 802A of the motor 802 and shrunk or tightened by the tightener 904A, 904B to clamp onto the end 802A of the motor 802, which in turn may clamp onto the body 502. In the illustrated embodiments, the band 902A of FIG. 9A may be a deformable metal or other material while the band 902B of FIG. 9B may be a hinged or clamshell band.

The tighteners 904A, 904B may be any tightener to tighten or shrink the band 902A, 902B. In the example of FIG. 9A, the tightener 904A includes a thumbscrew 906 inserted through holes defined in ends 908 of the band 902A combined with a nut 910 fixed to one of the bands ends 908. The thumbscrew 906 may be completely removed from the nut 910 and the ends 908 to, e.g., allow the band 902A to be opened and maneuvered into position, and then reinserted through the holes in the ends 908 and screwed into the nut 910. Screwing the thumbscrew 906 into the nut 910 may urge the ends 908 of the and 902A together which may shrink or tighten the band 902A, causing it to clamp onto the end 802A of the motor 802.

In the example of FIG. 9B, the tightener 904B includes a bolt 912 and a wing nut 914. The bolt 912 is rotatably coupled to one end 916 of the band 902B and thereby rotatable in a plane parallel to the generally circular opening of the band 902B. The other end 918 of the band 902B defines a slot 920 to receive the bolt 912. When the wing nut 914 is loose on the bolt 912, the bolt 912 may be rotated out of the slot 920 of the end 918, thereby freeing the band 902B to open at its hinge 922 and be maneuvered into position, e.g., on the end 802A of the motor 802. After being maneuvered into position, the band 902B may be closed as much as possible and the bolt 912 rotated into the slot 920 of the end 918, at which point the wing nut 914 may be screwed onto the bolt 912 to urge the ends 918, 916 of the band 902B together. The urging together of the ends 918, 916 may shrink or tighten the band 902B, causing it to clamp onto the end 802A of the motor 802.

Modifications, additions, or omissions may be made to FIGS. 9A and 9B without departing from the scope of the present disclosure. For example, the clamps 900A, 900B may include more or fewer elements depending on the implementation. Further, the clamps 900A, 900B may be configured to perform any number of operations as compared to those explicitly described.

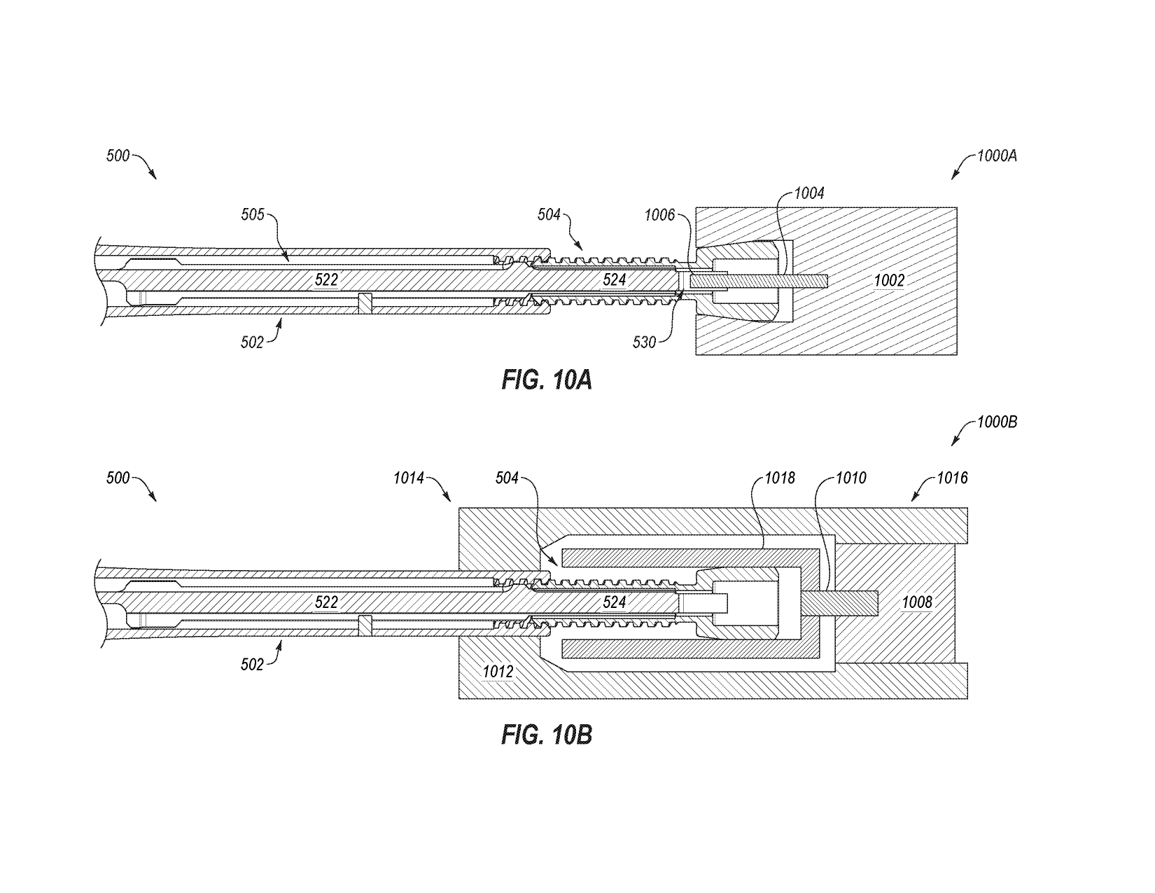

FIGS. 10A-10B include cross-sectional views of two example electric motor assemblies 1000A, 1000B that may be coupled to an IOL injection device, arranged in accordance with at least one embodiment herein. In FIGS. 10A-10B, the IOL injection device is depicted as the IOL injection device 500 of FIGS. 5A-5E but the electric motor assemblies 1000A, 1000B may alternatively or additionally be coupled to other IOL injection devices. Although not depicted in FIGS. 10A, and 10B, each of the electric motor assemblies 1000A, 1000B further includes or is coupled to a control device and a power supply and may optionally further include a communication interface.

The electric motor assembly 1000A of FIG. 10A includes an electric motor that includes both a stator 1002 and a drive shaft 1004 extending from and rotatably coupled to the stator 1002. In some embodiments, the stator 1002 of FIG. 10A may include, be included in, or correspond to the second component 406 of FIG. 4A or the stator 416 of FIG. 4B. Alternatively or additionally, the drive shaft 1004 may include, be included in, or correspond to the first component 404 of FIG. 4A or the drive shaft 418 of FIG. 4B.

The drive shaft 1004 includes a shaft end 1006 that is received in the slot 530 of the rod end 524 of the plunger rod 505. The shaft end 1006 may be inserted into the slot 530 to fix the drive shaft 1004 with respect to the plunger rod 505. In some embodiments, the shaft end 1006 includes thermoplastic polyurethane (TPU) and/or may be coupled to the plunger rod 505 by a friction fit between the TPU and the slot 530.

The stator 1002 is coupled to the adjustment knob 504 of the IOL injection device 500. As such, operation of the electric motor that includes the stator 1002 and the drive shaft 1004 may be effective to rotate the stator 1002 and the adjustment knob 504 relative to, e.g., the drive shaft 1004, the plunger rod 505, and the body 502. Further, and in view of the threaded engagement between the adjustment knob 504 and the body 502 and the axial engagement between the adjustment knob 504 and the plunger rod 505, rotation of the adjustment knob 504 relative to the body 502 may be effective to axially translate the plunger rod 505 relative to the body 502.

The stator 1002 may be coupled to the adjustment knob 504 in any suitable manner. For example, the stator 1002 may be clamped to the adjustment knob 504 using a clamp, such as either of the clamps 900A, 900B of FIGS. 9A and 9B or other suitable clamp. Alternatively or additionally, the stator 1002 may be coupled to the adjustment knob 504 using an adhesive, fastener, or other securing means. Alternatively or additionally, the stator 1002 may include inwardly directed blades, vanes, or ridges that have a complementary shape and size to engage the grooves 536 (not visible in FIG. 10A) of the adjustment knob 504.

By coupling the electric motor that includes the stator 1002 and the drive shaft 1004 only to the plunger rod 505 and the adjustment knob 504, the entire electric motor may translate axially together with the plunger rod 505 and the adjustment knob 504.

The electric motor assembly 1000B of FIG. 10B includes an electric motor that includes both a stator 1008 and a drive shaft 1010 extending from and rotatably coupled to the stator 1008. In some embodiments, the stator 1008 of FIG. 10B may include, be included in, or correspond to the first component 404 of FIG. 4A or the stator 416 of FIG. 4B. Alternatively or additionally, the drive shaft 1010 may include, be included in, or correspond to the second component 406 of FIG. 4A or the drive shaft 418 of FIG. 4B.

The motor assembly 1000B of FIG. 10B may further include a housing 1012 through which the stator 1008 is indirectly coupled to the body 502 of the IOL injection device 500. The housing 1012 may include, be included in, or correspond to the mechanical interface 414 of, e.g., FIGS. 4A and 4B. The housing 1012 may include a first end 1012A coupled to the body 502 of the IOL injection device 500. The housing 1012 may have a fixed angular orientation relative to the body 502 when the first end 1014 is coupled to the body 502. The first end 1014 of the housing 1012 may be coupled to the body 502 in any suitable manner. For example, the first end 1014 of the housing 1012 may be clamped to the body 502 using a clamp, such as either of the clamps 900A, 900B of FIGS. 9A and 9B or other suitable clamp. Alternatively or additionally, the first end 1014 of the housing 1012 may be coupled to the body 502 using an adhesive, fastener, or other securing means.

The housing 1012 may also include a second end 1016 opposite the first end 1014. The second end 1016 of the housing may house the electric motor that includes the stator 1008 and the drive shaft 1010. The second end 1016 of the housing 1012 may be coupled to the stator 1008 in any suitable manner. For example, the second end 1016 of the housing 1012 may be clamped to the stator 1008 using a clamp, such as either of the clamps 900A, 900B of FIGS. 9A and 9B, other suitable clamp, an adhesive, fastener, or other securing means.

The motor assembly 1000B of FIG. 10B may further include a turbine 1018 through which the drive shaft 1010 is indirectly coupled to the adjustment knob 504 of the IOL injection device 500. The turbine 1018 may include, be included in, or correspond to the mechanical interface 414 of, e.g., FIGS. 4A and 4B. The turbine 1018 may be coupled to the drive shaft 1010 and may be operably coupled to the adjustment knob 504 in any suitable manner. The adjustment knob 504 may be axially slidable along the turbine 1018 when operably coupled together and in response to rotation of the turbine 1018 and the adjustment knob 504.

In some embodiments, the turbine 1018 may include two or more blades, vanes, or ridges that extend axially and that have a complementary shape and size to engage the grooves 536 (not visible in FIG. 10B) of the adjustment knob 504. The adjustment knob 504 may be axially translatable relative to the body 502 of the IOL injection device 500 by a translatable distance. A length of the turbine 1018, and in particular of its blades, vanes, or ridges, may exceed the translatable distance to accommodate sliding of the adjustment knob 504 relative to the turbine 1018 through the entire translatable distance.

In the example of FIG. 10B, the stator 1008 is rotationally fixed with respect to the body 502 of the IOL injection device 500 via the housing 1012 while the drive shaft 1010 is rotationally fixed with respect to the adjustment knob 504 of the IOL injection device 500 via the turbine 1018. As such, operation of the electric motor that includes the stator 1008 and the drive shaft 1010 may be effective to rotate the drive shaft 1010, the turbine 1018, and the adjustment knob 504 relative to, e.g., the stator 1008, the housing 1012, and the body 502. Further, and in view of the threaded engagement between the adjustment knob 504 and the body 502 and the axial engagement between the adjustment knob 504 and the plunger rod 505, rotation of the adjustment knob 504 relative to the body 502 may be effective to axially translate the plunger rod 505 relative to the body 502. In addition, the axial length of the turbine 1018 may allow the turbine 1018 and the adjustment knob 504 to remain engaged with each other as the adjustment knob 504 translates axially and slides axially along and relative to the turbine 1018 notwithstanding the turbine 1018 being fixed in the axial direction with respect to the body 502.

Modifications, additions, or omissions may be made to FIGS. 10A and 10B without departing from the scope of the present disclosure. For example, the electric motor assemblies 1000A, 1000B may include more or fewer elements depending on the implementation. Further, the electric motor assemblies 1000A, 1000B may be configured to perform any number of operations as compared to those explicitly described.

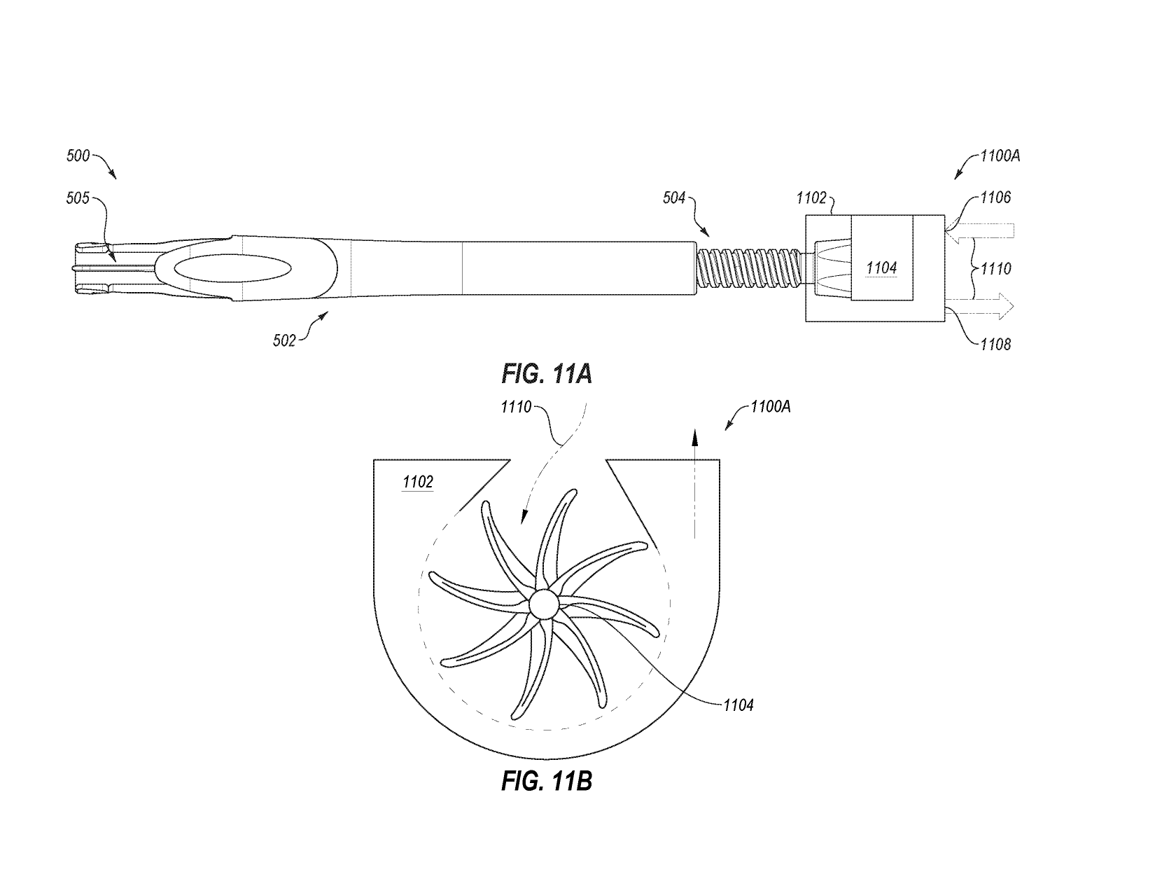

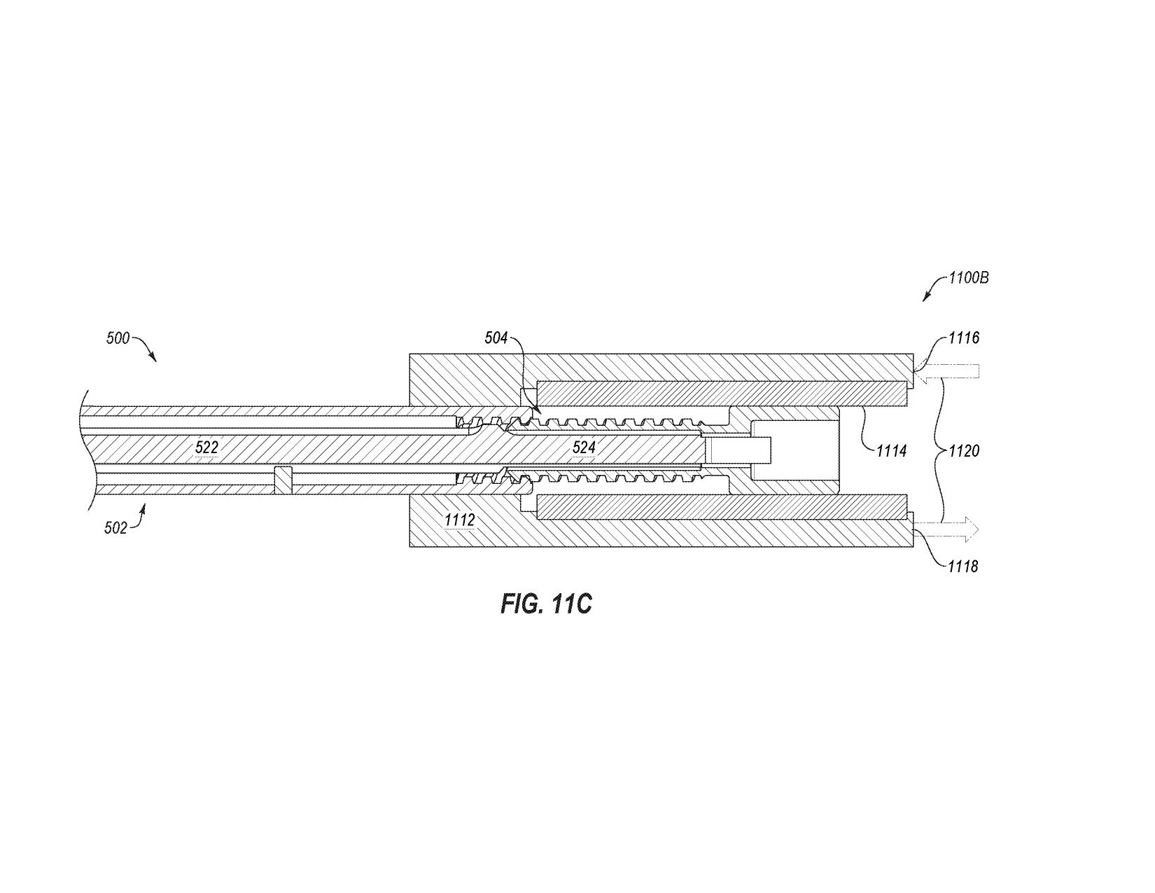

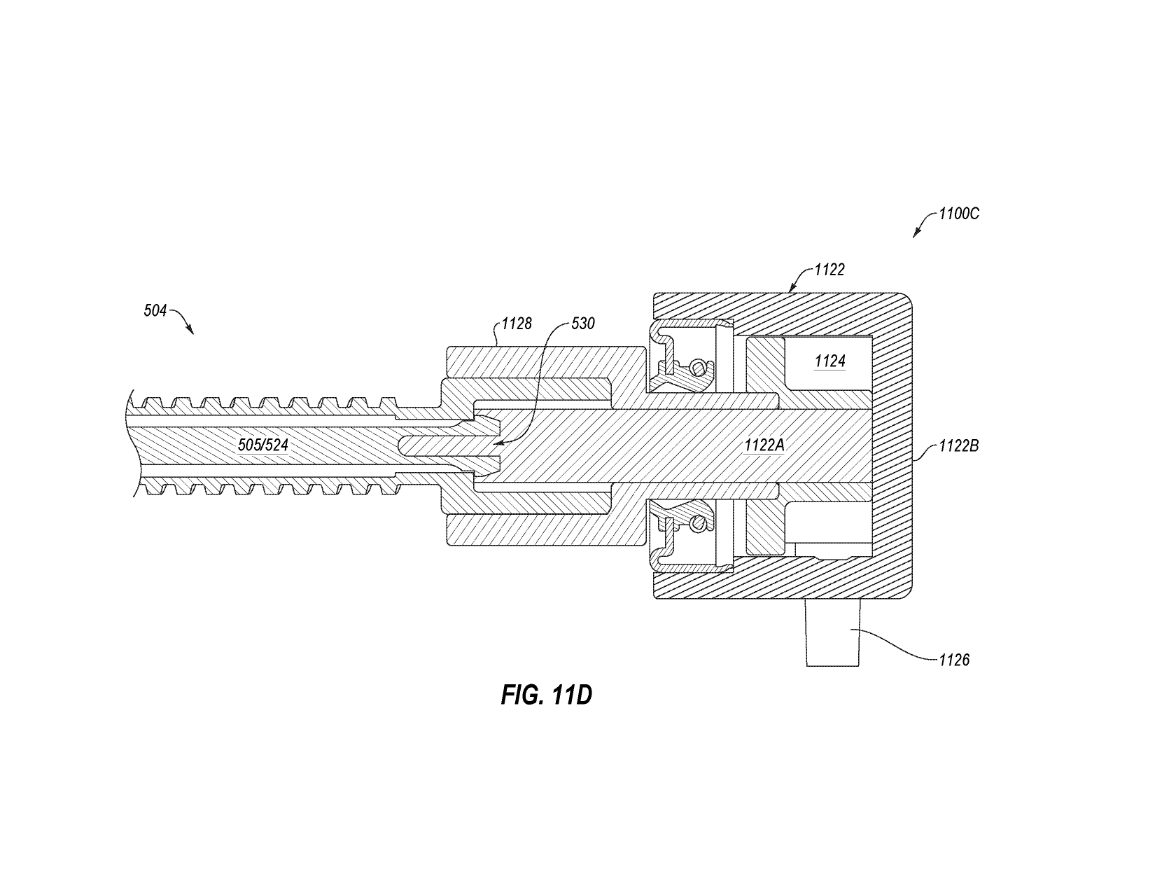

FIGS. 11A-11D illustrate various example hydraulic motor assemblies 1100A, 1100B, 1100C that may be coupled to an IOL injection device, arranged in accordance with at least one embodiment herein. In FIGS. 11A, 11C, and 11D, the IOL injection device is depicted as the IOL injection device 500 of FIGS. 5A-5E but the hydraulic motor assembly 1100A, 1100B, 1100C may alternatively or additionally be coupled to other IOL injection devices. Although not depicted in FIGS. 11A-11D, the hydraulic motor assembly 1100A, 1100B, 1100C further includes or is coupled to a control device and a power supply. By using a hydraulic motor (such as the hydraulic motor assemblies) 1100A, 1100B, 1100C, an electrical power source may be avoided and a surgical tool, such as a phacoemulsification surgical tool, which may already be present in the surgical suite may be used to power the motorized IOL delivery device.

The hydraulic motor assembly 1100A of FIG. 11A includes a hydraulic motor that includes both a stator 1102 and a rotor 1104 rotatably coupled to the stator 1102. In some embodiments, the stator 1102 of FIG. 11A may include, be included in, or correspond to the second component 406 of FIG. 4A or the stator 420 of FIG. 4C. Alternatively or additionally, the rotor 1104 may include, be included in, or correspond to the first component 404 of FIG. 4A or the rotor 422 of FIG. 4C.

As illustrated, the stator 1102 includes an input fluid port 1106 and an output fluid port 1108. The two ports 1106, 1108 may be coupled, e.g., via tubing 122, to the fluidics subsystem 120 of the console 106 of FIG. 1. Fluid may be forced into and through the hydraulic motor via the ports 1106, 1108 to cause the adjustment knob 504 to rotate and thereby axially translate the adjustment knob 504 and the plunger rod 505 relative to the body 502 of the IOL injection device 500. FIG. 11B illustrates an axial view of the hydraulic motor of the hydraulic motor assembly 1100A of FIG. 11A, arranged in accordance with at least one embodiment. As illustrated for a fluid path through the hydraulic motor, fluid 1110 may flow in through the input fluid port 1106, be directed through the hydraulic motor so as to flow across or past blades, fins, vanes, or the like of the rotor 1104 and thereby cause the rotor 1104 to rotate relative to the stator 1102. The fluid 1110 may then flow out through the output fluid port 1108. In some embodiments, the rotor 1104 may be fixed (e.g., with respect to the plunger rod 505 (not visible in FIG. 11B) of the IOL injection device 500) in which case the stator 1102 may rotate relative to the rotor 1104.

Returning to FIG. 11A, the rotor 1104 may be coupled to the plunger rod 505 (not visible in FIG. 11A) of the IOL injection device 500. For example, the rotor 1104 may have a drive shaft extending therefrom with a shaft end that is received in the slot 530 (not visible in FIG. 11A) of the rod end 524 (not visible in FIG. 11A) of the plunger rod 505. The shaft end of the rotor 1104 may be inserted into the slot 530 to fix the rotor 1104 with respect to the plunger rod 505. In some embodiments, the shaft end includes TPU and/or may be coupled to the plunger rod 505 by a friction fit between the TPU and the slot 530.

The stator 1102 is coupled to the adjustment knob 504 of the IOL injection device 500. As such, operation of the hydraulic motor that includes the stator 1102 and the rotor 1104 may be effective to rotate the stator 1102 and the adjustment knob 504 relative to, e.g., the rotor 1104, the plunger rod 505, and the body 502. Further, and in view of the threaded engagement between the adjustment knob 504 and the body 502 and the axial engagement between the adjustment knob 504 and the plunger rod 505, rotation of the adjustment knob 504 relative to the body 502 may be effective to axially translate the plunger rod 505 relative to the body 502.

The stator 1102 may be coupled to the adjustment knob 504 in any suitable manner. For example, the stator 1102 may be clamped to the adjustment knob 504 using a clamp, such as either of the clamps 900A, 900B of FIGS. 9A and 9B or other suitable clamp. Alternatively or additionally, the stator 1102 may be coupled to the adjustment knob 504 using an adhesive, fastener, or other securing means. Alternatively or additionally, the stator 1102 may include inwardly directed blades, vanes, or ridges that have a complementary shape and size to engage the grooves 536 (not visible in FIG. 11A) of the adjustment knob 504.

Although not illustrated in FIG. 11A, the hydraulic motor assembly 1100A may further include one or more O-rings or other structure to seal the fluid 1110 within the hydraulic motor and inhibit leaks.

By coupling the hydraulic motor that includes the stator 1102 and the rotor 1104 only to the plunger rod 505 and the adjustment knob 504, the entire hydraulic motor may translate axially together with the plunger rod 505 and the adjustment knob 504.

The hydraulic motor assembly 1100B of FIG. 11C includes a hydraulic motor that includes both a stator 1112 and a rotor 1114 rotatably coupled to the stator 1112. In some embodiments, the stator 1112 of FIG. 11C may include, be included in, or correspond to the first component 404 of FIG. 4A or the stator 416 of FIG. 4B. Alternatively or additionally, the rotor 1114 may include, be included in, or correspond to the second component 406 of FIG. 4A or the rotor 422 of FIG. 4C.

In FIG. 11C, the stator 1112 is coupled to the body 502 of the IOL injection device 500. The stator 1112 may be coupled to the body 502 in any suitable manner. For example, the stator 1112 may be clamped to the body 502 using a clamp, such as either of the clamps 900A, 900B of FIGS. 9A and 9B or other suitable clamp. Alternatively or additionally, the stator 1112 may be coupled to the body 502 using an adhesive, fastener, or other securing means.

Similar to the stator 1102 of FIG. 11A, the stator 1112 of FIG. 11C includes an input fluid port 1116 and an output fluid port 1118. The two ports 1116, 1118 may be coupled, e.g., via tubing 122, to the fluidics subsystem 120 of the console 106 of FIG. 1. Fluid 1120 may be forced into and through the hydraulic motor via the ports 1116, 1118 to cause the adjustment knob 504 to rotate and thereby axially translate the adjustment knob 504 and the plunger rod 505 relative to the body 502 of the IOL injection device 500. For example, the fluid 1120 may flow across blades, vanes, or the like mechanically coupled to the stator 1112, thereby causing the stator 1112 to rotate relative to the rotor 1114.

The rotor 1114 is coupled to the adjustment knob 504 of the IOL injection device 500. The adjustment knob 504 may be axially slidable along the rotor 1114 when operably coupled together and in response to rotation of the rotor 1114 and the adjustment knob 504.

In some embodiments, the rotor 1114 may include two or more blades, vanes, or ridges that extend axially and that have a complementary shape and size to engage the grooves 536 (not visible in FIG. 11C) of the adjustment knob 504. The adjustment knob 504 may be axially translatable relative to the body 502 of the IOL injection device 500 by a translatable distance. A length of the rotor 1114, and in particular of its blades, vanes, or ridges, may exceed the translatable distance to accommodate sliding of the adjustment knob 504 relative to the rotor 1114 through the entire translatable distance.

In the example of FIG. 11C, the stator 1112 is rotationally fixed with respect to the body 502 of the IOL injection device 500 while the rotor 1114 is rotationally fixed with respect to the adjustment knob 504 of the IOL injection device 500. As such, operation of the hydraulic motor that includes the stator 1112 and the rotor 1114 may be effective to rotate the rotor 1114 and the adjustment knob 504 relative to, e.g., the stator 1112 and the body 502. Further, and in view of the threaded engagement between the adjustment knob 504 and the body 502 and the axial engagement between the adjustment knob 504 and the plunger rod 505, rotation of the adjustment knob 504 relative to the body 502 may be effective to axially translate the plunger rod 505 relative to the body 502. In addition, the axial length of the rotor 1114 may allow the rotor 1114 and the adjustment knob 504 to remain engaged with each other as the adjustment knob 504 translates axially and slides axially along and relative to the rotor 1114 notwithstanding the rotor 1114 being fixed in the axial direction with respect to the body 502.

The hydraulic motor assembly 1100C of FIG. 11D includes a hydraulic motor that includes both a stator 1122 and a rotor 1124 rotatably coupled to the stator 1122. In some embodiments, the stator 1122 of FIG. 11C may include, be included in, or correspond to the first component 404 of FIG. 4A or the stator 416 of FIG. 4B. Alternatively or additionally, the rotor 1124 may include, be included in, or correspond to the second component 406 of FIG. 4A or the rotor 422 of FIG. 4C.

In FIG. 11D, the stator 1122 is coupled to the plunger rod 505 of the IOL injection device 500. As illustrated, the stator 1122 has a shaft 1122A extending from its base 1122B with a shaft end that is received in the slot 530 of the rod end 524 of the plunger rod 505. The shaft end of the shaft 1122A may be inserted into the slot 530 to fix the stator 1122 with respect to the plunger rod 505. In some embodiments, the shaft end of the shaft 1122A includes TPU and/or may be coupled to the plunger rod 505 by a friction fit between the TPU and the slot 530. Alternatively or additionally, the shaft end of the shaft 1122A may be coupled to the plunger rod 505 (e.g., in the slot 530) using an adhesive or other securing means.

Similar to the stator 1102, 1112 of FIGS. 11A-11C, the stator 1122 of FIG. 11D includes an input fluid port 1126 and an output fluid port (not visible in FIG. 11D). The two ports may be coupled, e.g., via tubing 122, to the fluidics subsystem 120 of the console 106 of FIG. 1. Fluid may be forced into and through the hydraulic motor via the ports to cause the adjustment knob 504 to rotate and thereby axially translate the adjustment knob 504 and the plunger rod 505 relative to the body 502 (not visible in FIG. 11D) of the IOL injection device 500. For example, the fluid may flow across blades, vanes, or the like mechanically coupled to the rotor 1124, thereby causing the rotor 1124 to rotate relative to the stator 1122.

The rotor 1124 is coupled to the adjustment knob 504 of the IOL injection device 500 through a coupler 1128. The coupler 1128 may, in effect, extend the rotor 1124 out of the base 1122B of the stator 1122 to couple to the adjustment knob 504. The shaft 1122A of the stator 1122 may pass through each of the rotor 1124 and the coupler 1128 such that the rotor 1124 and the coupler 1128 may rotate around the shaft 1122A relative to the stator 1122, e.g., when fluid passes over blades, vanes, or the like mechanically coupled to the rotor 1124.

In some embodiments, the coupler 1128 may include two or more blades, vanes, or ridges that extend axially and that have a complementary shape and size to engage the grooves 536 (not visible in FIG. 11D) of the adjustment knob 504. Alternatively or additionally, the coupler 1128 may be clamped to the adjustment knob 504 using a clamp, such as either of the clamps 900A, 900B of FIGS. 9A and 9B or other suitable clamp. Alternatively or additionally, the coupler 1128 may be coupled to the adjustment knob 504 using an adhesive, fastener, or other securing means.

In the example of FIG. 11D, the stator 1122 is rotationally fixed with respect to the plunger rod 505 and therefore the body 502 of the IOL injection device 500 while the rotor 1124 and the coupler 1128 are rotationally fixed with respect to the adjustment knob 504 of the IOL injection device 500. As such, operation of the hydraulic motor that includes the stator 1122 and the rotor 1124 may be effective to rotate the rotor 1124 and the adjustment knob 504 relative to the stator 1122, the plunger rod 505, and the body 502. Further, and in view of the threaded engagement between the adjustment knob 504 and the body 502 and the axial engagement between the adjustment knob 504 and the plunger rod 505, rotation of the adjustment knob 504 relative to the body 502 may be effective to axially translate the plunger rod 505 relative to the body 502.

Although not illustrated in FIG. 11D, the hydraulic motor assembly 1100C may further include one or more O-rings or other structure to seal fluid within the hydraulic motor and inhibit leaks.

By coupling the hydraulic motor that includes the stator 1122 and the rotor 1124 only to the plunger rod 505 and the adjustment knob 504, the entire hydraulic motor may translate axially together with the plunger rod 505 and the adjustment knob 504.

Modifications, additions, or omissions may be made to FIGS. 11A-11D without departing from the scope of the present disclosure. For example, the hydraulic motor assemblies 1100A, 1100B, 1100C may include more or fewer elements depending on the implementation. Further, the hydraulic motor assemblies 1100A, 1100B, 1100C may be configured to perform any number of operations as compared to those explicitly described.

An example method of using an IOL injection device with an electric motor assembly will now be described. The IOL injection device may include the IOL injection device 104, 500 of FIGS. 1 and 5A-5E, for example. The electric motor assembly may include the motor assembly 400A, 400B, 1000A, 1000B of FIGS. 4A, 4B, and 10A-10B, for example. The method may include removably coupling a stator of an electric motor of the electric motor assembly to an adjustment knob or body of the IOL injection device. The method may include removably coupling a drive shaft of the electric motor to a plunger rod of the IOL injection device or the adjustment knob. The method may include receiving operator input at a control device coupled to the electric motor to initiate rotation between the stator and the drive shaft of the electric motor. The method may include rotating the adjustment knob responsive to the relative rotation between the stator and the drive shaft. The method may include axially translating the adjustment knob and the plunger rod responsive to rotation of the adjustment knob. Receiving the operator input at the control device may include receiving input effective to slide a button coupled to a potentiometer (or encoder or other device) of the control device relative to the body. Sliding the button may turn the motor on (or off) and/or control a rotational speed of the adjustment knob. Alternatively or additionally, receiving the operator input at the control device may include receiving input effective to operate a foot pedal coupled to the electric motor through a phacoemulsification surgical console. Operating the foot pedal may turn the motor on (or off) and/or control a rotational speed of the adjustment knob.

An example method of using an IOL injection device with a hydraulic motor assembly will now be described. The IOL injection device may include the IOL injection device 104, 500 of FIGS. 1 and 5A-5E, for example. The hydraulic motor assembly may include the motor assembly 400A, 400C, 1100A, 1100B, 1100C of FIGS. 4A, 4C, and 11A-11D, for example. The method may include removably coupling a stator of a hydraulic motor of the hydraulic motor assembly to an adjustment knob, body, or plunger rod of the IOL injection device. The method may include removably coupling a rotor of the hydraulic motor to a plunger rod of the IOL injection device or the adjustment knob. The method may include receiving operator input at a control device coupled to the hydraulic motor to initiate relative rotation between the stator and the rotor of the hydraulic motor. The method may include rotating the adjustment knob responsive to the relative rotation between the stator and the rotor. The method may include axially translating the adjustment knob and the plunger rod responsive to rotation of the adjustment knob. Receiving the operator input at the control device may include receiving input effective to operate a foot pedal coupled to the hydraulic motor through a phacoemulsification surgical console. Operating the foot pedal may turn the motor on (or off) and/or control a rotational speed of the adjustment knob. In some embodiments, the method may further include, prior to receiving the operator input, fluidly coupling the hydraulic motor to a fluid supply, the fluid supply including a phacoemulsification surgical console. Fluidly coupling the hydraulic motor to the fluid supply may include coupling input and output fluid ports of the hydraulic motor assembly to a disposable cassette coupled to the phacoemulsification surgical console.

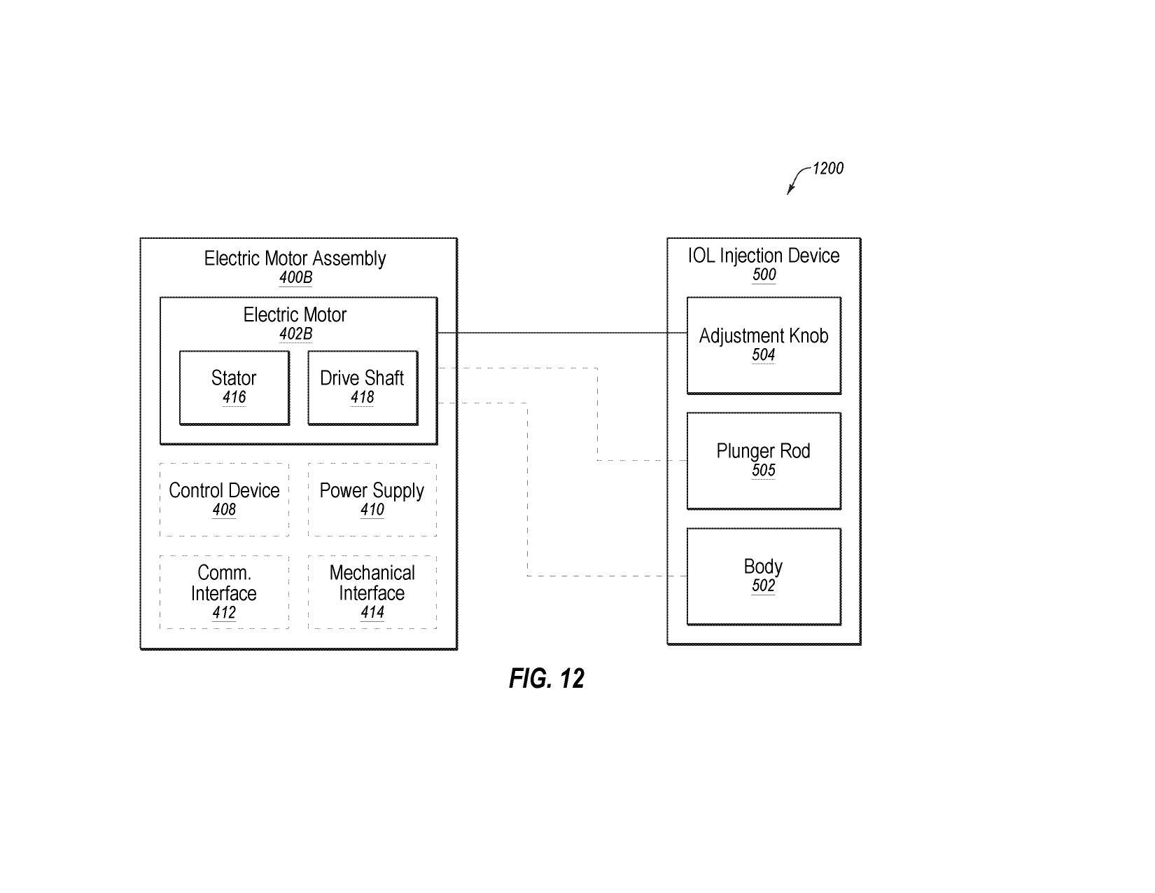

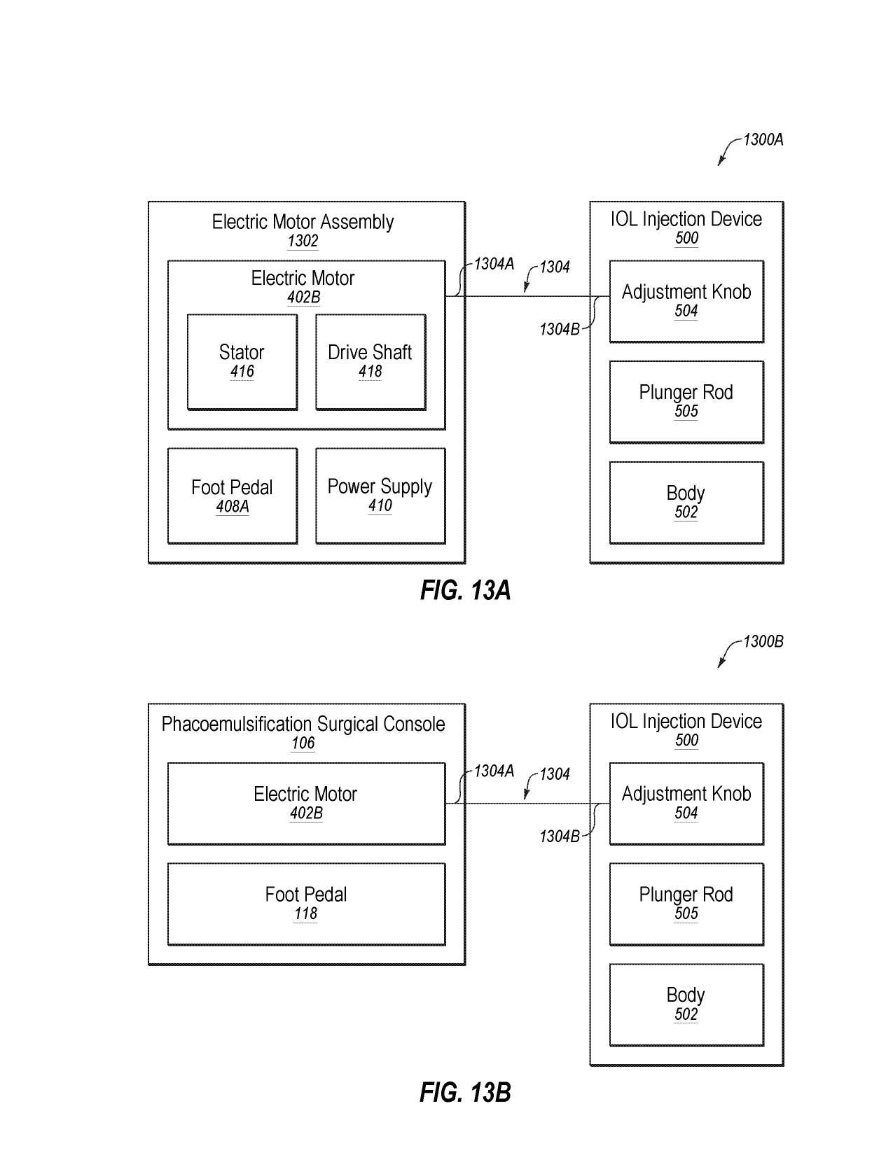

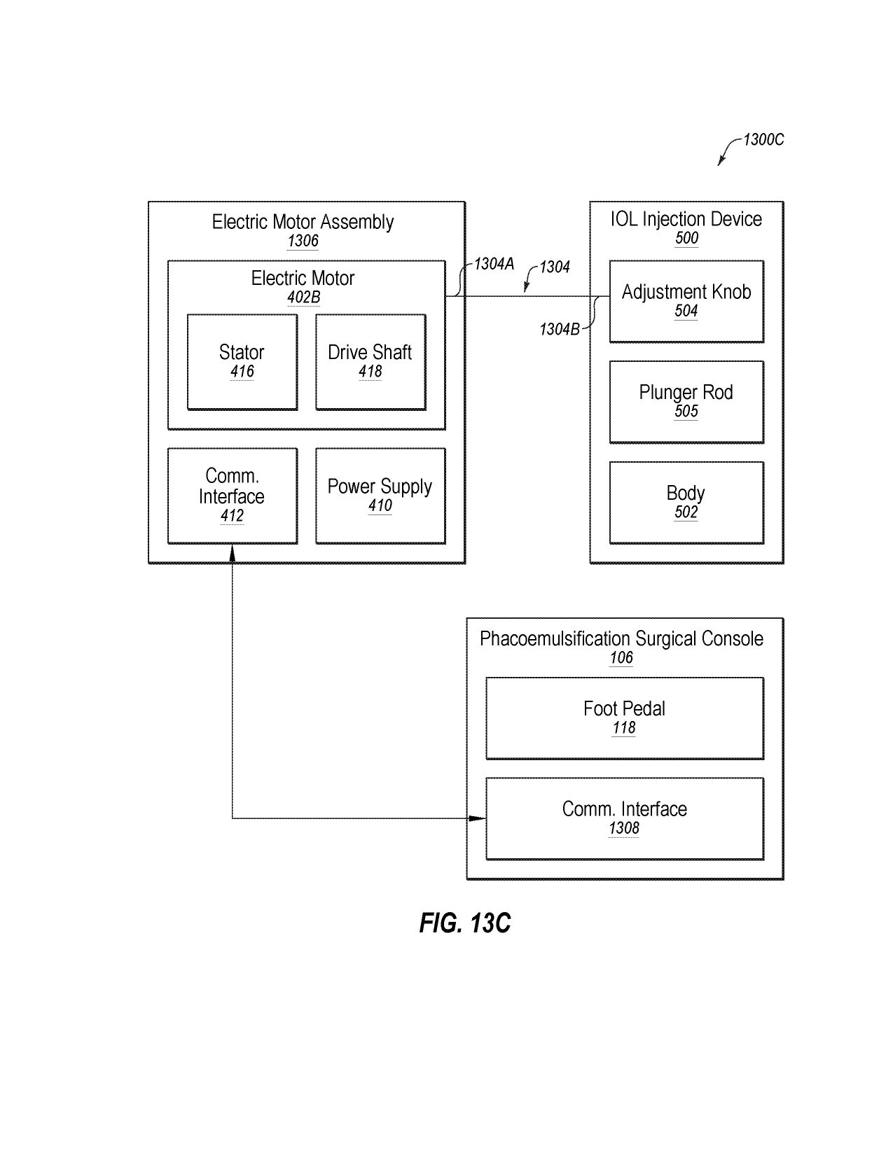

FIG. 12 illustrates an example IOL injection system 1200 that includes an assembly operable with an IOL injection device, arranged in accordance with at least one embodiment herein. In the illustrated example, the assembly includes the electric motor assembly 400B of FIG. 4B (or other assemblies herein) while the IOL injection device includes the IOL injection device 500 of FIGS. 5A-5D (or other IOL injection devices). Various other example assemblies are described elsewhere herein, including with respect to FIGS. 13A-13C.

The electric motor assembly 400B includes the electric motor 402B that includes the stator 416 and the drive shaft 418. The electric motor 402B is configured to generate rotational motion, e.g., of the drive shaft 418 relative to the stator 416 and/or of the stator 416 relative to the drive shaft 418. The stator 416 or the drive shaft 418 may be removably couplable to the adjustment knob 504 of the IOL injection device 500, e.g., via the mechanical interface 414. Although not required, in some embodiments, the other of the drive shaft 418 or the stator 416 may be removably couplable to the plunger rod 505 and/or the body 502 of the IOL injection device 500.

The assembly of FIG. 12 further includes the control device 408, which may be included as part of the electric motor assembly 400B or externally thereto. The control device 408 is generally configured to control operation of the electric motor 402B, such as starting and stopping rotation of the electric motor 402B, controlling a rotational speed of the electric motor 402B, and/or controlling a rotational direction of the electric motor 402B. As already discussed elsewhere herein, the control device 408 may include a foot pedal or other suitable control device. In some embodiments, the control device 408 is configured to provide variable rotational speed control and/or bidirectional rotation control of the electric motor 402B.

The stator 416 or the drive shaft 418 may be configured to rotate the adjustment knob 504 of the IOL injection device 500 relative to the body 502 and the plunger rod 505 of the IOL injection, e.g., to axially advance or retract the plunger rod 505 relative to the body 502 of the IOL injection device 500 and/or a corresponding cartridge (e.g., the cartridge 700 of FIG. 7).

In an example configuration, the mechanical interface 414 may include a flex shaft cable with a first end coupled to the drive shaft 418 and a second end coupled or couplable to the adjustment knob 504 of the IOL injection device 500. The flex shaft cable may be configured to transmit the rotational motion generated by the electric motor 402B from the electric motor 402B to the adjustment knob 500. In this example, a surgeon may hold and retain, e.g., the body 502 of the IOL injection device 500 in one of the surgeon’s hands such that the adjustment knob 504 may rotate relative to the body 502 and the plunger rod 505 when the rotational motion is transmitted from the electric motor 402B through the flex shaft cable to the adjustment knob 504.

In another example configuration corresponding to FIG. 10A, the stator 416 may be coupled or couplable to the adjustment knob 504 with a fixed angular orientation relative to the adjustment knob 504. The drive shaft 418 may be coupled or couplable to the plunger rod 505 such that the drive shaft 418 is angularly fixed relative to the plunger rod 505 and the body 502. In such a configuration, the rotational motion generated by the electric motor 402 may cause the stator 416 and the adjustment knob 504 to rotate relative to the drive shaft 418, the plunger rod 505, and the body 502 as described with respect to FIG. 10A.

In another example configuration corresponding to FIG. 10B, the stator 416 may be coupled or couplable to the body 502, e.g., via a housing (similar to the housing 1012 of FIG. 10B) with a fixed angular orientation relative to the body 502. The drive shaft 418 may be coupled or couplable to the adjustment knob 504, e.g., via a turbine (similar to the turbine 1018 of FIG. 10B), with a fixed angular orientation relative to the adjustment knob 504. The housing and/or the turbine may be included as part of the mechanical interface 414. In such a configuration, the rotational motion generated by the electric motor 402 may cause the drive shaft 418, the turbine, and the adjustment knob 504 to rotate relative to the stator 416, the housing, the plunger rod 505, and the body 502 as described with respect to FIG. 10B.

FIGS. 13A-13C illustrate various example IOL injection systems 1300A-1300C that each includes an assembly operable with an IOL injection device, arranged in accordance with at least one embodiment herein. In the illustrated examples, each assembly may include at least some portions of the electric motor assembly 400B of FIG. 4B or other assemblies herein while the IOL injection device may include the IOL injection device 500 of FIGS. 5A-5D or other IOL injection device.

In FIG. 13A, the assembly includes the electric motor 402B and a control device in the form of a foot pedal 408A. The electric motor 402B and the foot pedal 408A may be part of a standalone electric motor assembly 1302 that further includes the power supply 410 of FIGS. 4B and 12 integrated with the electric motor assembly 1302.

The foot pedal 408A may be configured to receive user input to control at least one of initiation of rotation of the electric motor 402B, termination of rotation of the electric motor 402B, a rotational speed of the electric motor 402B, or a rotational direction of the electric motor 402B. In an example implementation, the foot pedal 408A may be rotatably coupled to a base and biased to a neutral position in which no rotation is generated by the electric motor 402B. Rotating the foot pedal 408A from the neutral position in a first foot pedal direction may initiate rotation of the electric motor 402B in a first electric motor direction. Increasing the amount of rotation of the foot pedal 408A in the first foot pedal direction may increase the rotational speed of the electric motor 402B in the first electric motor direction. Removing force from the foot pedal 408A with the foot pedal 408A initially rotated in the first foot pedal direction may allow the foot pedal 408A to return to the neutral position, allowing the electric motor 402B to decrease its rotational speed in the first electric motor direction and eventually come to a stop. Rotating the foot pedal 408A from the neutral position in a second foot pedal direction opposite the first food pedal direction may initiate rotation of the electric motor 402B in a second electric motor direction that is opposite the first electric motor direction. Increasing the amount of rotation of the foot pedal 408A in the second foot pedal direction may increase the rotational speed of the electric motor 402B in the second electric motor direction. Removing force from the foot pedal 408A with the foot pedal 408A initially rotated in the second foot pedal direction may allow the foot pedal 408A to return to the neutral position, allowing the electric motor 402B to decrease its rotational speed in the second electric motor direction and eventually come to a stop.

In some embodiments, other configurations of the foot pedal 408A are also contemplated. For example, the foot pedal 408A may include multiple pedals or buttons on a given interface. A first pedal may be depressed to cause a corresponding speed of rotation of the electric motor 402B that is proportional to the amount of depression of the first pedal. The default direction of rotation may correspond to a forward direction of the plunger rod 505 into the body 502. A secondary button or pedal may be pressed to change the direction of rotation, followed by depressing the first pedal to cause a corresponding speed of rotation of the electric motor 402B that is in the opposite direction, such as in a reverse direction of the plunger rod 505 out of the body 502. As another example, the foot pedal 408A may include multiple pedals or buttons on a given interface. A first pedal may be depressed to cause a corresponding (e.g., proportional) speed of rotation of the electric motor 402B that corresponds to the amount of depression of the first pedal. The first pedal may cause a direction of rotation that may correspond to a forward direction of the plunger rod 505 into the body 502. A secondary pedal may be depressed to cause a corresponding (e.g., proportional) speed of rotation of the electric motor 402B that is in the opposite direction, such as in a reverse direction of the plunger rod 505 out of the body 502.

The assembly of FIG. 13A further includes a flex shaft cable 1304 that may include, be included in, or otherwise correspond to at least a portion of the mechanical interface 414 of FIGS. 4B and 12. A first end 1304A of the flex shaft cable 1304 may be coupled or couplable to the drive shaft 418. A second end 1304B of the flex shaft cable 1304 may be coupled or couplable to the adjustment knob 504 of the IOL injection device 504. In general, the flex shaft cable 1304 may be configured to transmit rotational motion from the electric motor 402B to the adjustment knob 504.

A first connector (not shown in FIG. 13A) at the first end 1304A of the flex shaft cable 1304 may be configured to couple the first end 1304A of the flex shaft cable 1304 to the electric motor 402B, e.g., to the drive shaft 418 in some embodiments. A second connector (not shown in FIG. 13A) at the second end 1304B of the flex shaft cable 1304 may be configured to couple the second end 1304B of the flex shaft cable 1304 to the adjustment knob 504. Each of the first and second connectors may be configured to detachably couple the respective first end 1304A or second end 1304B of the flex shaft cable 1304 to the respective electric motor 402B or the adjustment knob 504. Alternatively or additionally, each of the first and second connectors may include, e.g., a clamp (such as clamps 900A, 900B of FIGS. 9A-9B), a nut, a bolt, an adhesive strip, a detent connector (e.g., ball in a divot that allows push on/pull off functionality), a snap-fit connector, or other suitable connector. The flex shaft cable 1304, the first connector, and the second connector collectively form an assembly that may be operable with the IOL injection device 500 in, e.g., the configurations of FIGS. 13A-13C.

The flex shaft cable 1304 may include a sterilizable outer sheath. Alternatively or additionally, the flex shaft cable 1304 itself may be sterilizable. In these and other embodiments, the outer sheath and/or the flex shaft cable 1304 may be sterilized in advance of use of the system 1300.

In FIG. 13B, the assembly includes the electric motor 402B and a control device in the form of the foot pedal 118 of FIGS. 1-2. In the example of FIG. 13B, the electric motor 402B and the foot pedal 118 are part of the console 106. The electric motor 402B may be a dedicated electric motor to control operation of the IOL injection device 500, e.g., to rotate the adjustment knob 504 relative to the plunger rod 505 and the body 502.

Alternatively, the electric motor 402B may have multiple uses. For example, the electric motor 402B may selectively control operation of the adjustment knob 504 of the IOL injection device 500 or of a peristaltic pump (or other device) of the console 106. In some embodiments, a user interface of the console 106 may be used to switch the electric motor 402B between control of the adjustment knob 504 and the peristaltic pump. The user interface may include an electronic control that a user selects, or a mechanical switch that changes or adjusts the physical connection.

As in FIG. 13A, in FIG. 13B the flex shaft cable 1304 is coupled or couplable to the electric motor 402B at the first end 1304A and coupled or couplable to the adjustment knob 504 at the second end 1304B. The first and second connectors discussed with respect to FIG. 13A may be used to detachably couple the first or second end 1304A, 1304B to the corresponding electric motor 402B (e.g., drive shaft 418) or adjustment knob 504.

In FIG. 13C, the assembly includes the electric motor 402B and a control device in the form of the foot pedal 118 of the console 106. The electric motor 402B may be part of a standalone electric motor assembly 1306 that further includes the power supply 410 and the communication interface 412 of FIGS. 4B and 12 integrated with the electric motor assembly 1306.

In this example, the console 106 may further include a communication interface 1308 to communicatively couple the foot pedal 118 to the electric motor 402B through the communication interface 412. For example, each of the communication interface 412 of the electric motor assembly 1306 and the communication interface 1308 of the console 106 may include a Bluetooth chip and the two may be paired to relay commands from the foot pedal 118 to the electric motor 402B. While Bluetooth is provided as an example, it will be appreciated that any wireless or wired communication protocols or modalities are contemplated, such as WiFi, Zigbee, Near-Field Communications (NFC), etc.

As in FIG. 13A, in FIG. 13C the flex shaft cable 1304 is coupled or couplable to the electric motor 402B at the first end 1304A and coupled or couplable to the adjustment knob 504 at the second end 1304B. The first and second connectors discussed with respect to FIG. 13A may be used to detachably couple the first or second end 1304A, 1304B to the corresponding electric motor 402B (e.g., drive shaft 418) or adjustment knob 504.

An example method of delivering an IOL, e.g., using any of the assemblies of FIGS. 12-13C, will now be described. The method may include attaching a connector to an adjustment knob of an IOL injection device, the connector coupled to a flex shaft cable. For example, the second connector discussed in connection with FIGS. 13A-13C coupled to the second end 1304B of the flex shaft cable 1304 may be attached to the adjustment knob 504 of the IOL injection device 500. The method may include activating an electric motor mechanically coupled to the flex shaft cable to generate rotational motion. For example, the electric motor 402B mechanically coupled to the first end 1304A of the flex shaft cable 1304, e.g., using the first connector discussed in connection with FIGS. 13A-13C, may be activated, e.g., responsive to a signal from the foot pedal 408A, 118, to generate rotational motion between the stator 416 and the drive shaft 418. The method may include transmitting rotational motion from the electric motor through the flex shaft cable and the connector to the adjustment knob. The method may include rotating the adjustment knob of the IOL injection device to advance an IOL relative to the IOL injection device. The method may further include controlling the electric motor via a foot pedal. Controlling the electric motor may include controlling a rotational speed or rotational direction of the electric motor. The method may further include sterilizing the flex shaft cable, or an outer sheath of the flex shaft cable, and the connector prior to the attaching.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B. Additionally, use of the term “based on” should not be interpreted as “only based on” or “based only on.” Rather, a first element being “based on” a second element includes instances in which the first element is based on the second element but may also be based on one or more additional elements.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 2) or examples, or combined with aspects of another example. The following sets of examples are non-limiting summaries of some example implementations presented herein. When referencing an example (e.g., example 1), that reference is specifically to the corresponding example within the same example set and example embodiment, although such example may also be applicable to the other example sets and embodiments.

### Example Set 1

### ELECTRIC MOTOR EXAMPLE EMBODIMENTS

Example 1. A motor assembly for an intraocular lens (IOL) injection device, the motor assembly comprising:

a motor couplable to a power supply and a control device;

a first component removably couplable to a first portion of the IOL injection device, the first component of the motor assembly having a fixed angular orientation relative to the first portion of the IOL injection device when coupled to the first portion of the IOL injection device; and

a second component removably couplable to a second portion of the IOL injection device that is rotatable relative to the first portion of the IOL injection device,

wherein the second component is drivable by the motor to rotate the second portion of the IOL injection device relative to the first portion of the IOL injection device.

Example 2. The motor assembly of any example herein, particularly of example 1, wherein:

the motor comprises a stator and a drive shaft;

the first component of the motor assembly comprises the stator; and

the second component of the motor assembly comprises the drive shaft.

Example 3. The motor assembly of any example herein, particularly of example 1, wherein:

the motor comprises a stator and a drive shaft;

the first component of the motor assembly comprises the drive shaft; and

the second component of the motor assembly comprises the stator.

Example 4. The motor assembly of any example herein, particularly of example 3, wherein:

the first portion of the IOL injection device to which the drive shaft of the motor assembly is removably couplable comprises a plunger rod of the IOL injection device;

the plunger rod of the IOL injection device is angularly fixed relative to a body of the IOL injection device;

the second portion of the IOL injection device to which the stator of the motor assembly is removably couplable comprises an adjustment knob of the IOL injection device;

the adjustment knob of the IOL injection device is rotatable by the motor relative to the body and the plunger rod of the IOL injection device; and

rotation of the adjustment knob relative to the body and the plunger rod is effective to axially translate the plunger rod relative to the body.

Example 5. The motor assembly of any example herein, particularly of example 1, wherein:

the motor comprises a stator and a drive shaft;

the stator is couplable to the first component and has a fixed angular orientation relative to the first portion of the IOL injection device; and

the drive shaft is couplable to the second component and is rotatable relative to the first portion of the IOL injection device.

Example 6. The motor assembly of any example herein, particularly of example 1, wherein:

the first portion of the IOL injection device comprises a body of the IOL injection device;

the first component of the motor assembly comprises a housing that has a first end coupled to the body of the IOL injection device and a second end that houses the motor;

the second component of the motor assembly comprises a turbine coupled to and rotatable by the motor, the turbine extending axially a first distance equal to or greater than a translatable distance of the second portion of the IOL injection device relative to the first portion of the IOL injection device.

Example 7. The motor assembly of any example herein, particularly of any one of examples 1-6, further comprising the power supply, wherein the power supply comprises a battery.

Example 8. The motor assembly of any example herein, particularly of any one of examples 1-7, further comprising the control device, wherein the control device comprises a potentiometer coupled to the motor and a button mountable to the IOL injection device, the button operably coupled to the potentiometer and operable by a user to adjust a rotational speed of the motor.

Example 9. The motor assembly of any example herein, particularly of any one of examples 1-8, wherein the control device is wirelessly coupled to the motor.

Example 10. The motor assembly of any example herein, particularly of any one of examples 1-8, wherein the control device is hardwired to the motor.

Example 11. The motor assembly of any example herein, particularly of any one of examples 1-6, wherein the power supply comprises a phacoemulsification surgical console and the control device comprises a foot pedal of the phacoemulsification surgical console.

Example 12. An electric motor assembly removably couplable to an IOL injection device, the electric motor assembly comprising:

an electric motor couplable to a power supply and a control device, the electric motor comprising:

a stator; and

a drive shaft;

wherein:

the stator is couplable to an adjustment knob of the IOL injection device with a fixed angular orientation relative to the adjustment knob and the drive shaft is couplable to a plunger rod of the IOL injection device with a fixed angular orientation relative to the plunger rod; or

the stator is couplable to a body of the IOL injection device with a fixed angular orientation relative to the body and the drive shaft is couplable to the adjustment knob with a fixed angular orientation relative to the adjustment knob.

Example 13. The electric motor assembly of any example herein, particularly of example 12, wherein:

the stator is couplable to the adjustment knob;

the drive shaft is couplable to the plunger rod;

operation of the electric motor is effective to rotate the stator and the adjustment knob relative to the plunger rod and the body; and

rotation of the adjustment knob relative to the body is effective to axially translate the plunger rod relative to the body.

Example 14. The electric motor assembly of any example herein, particularly of example 12 or 13, wherein the plunger rod includes a rod end that defines a slot and the drive shaft includes a shaft end receivable in the slot.

Example 15. The electric motor assembly of any example herein, particularly of example 14, wherein the shaft end comprises TPU and the shaft end of the drive shaft is couplable to the slot of the rod end by a friction fit between the TPU and the slot.

Example 16. The electric motor assembly of any example herein, particularly of example 12, wherein:

the stator is couplable to the body;

the drive shaft is couplable to the adjustment knob;

operation of the electric motor is effective to rotate the drive shaft and the adjustment knob relative to the plunger rod and the body; and

rotation of the adjustment knob relative to the body is effective to axially translate the plunger rod relative to the body.

Example 17. The electric motor assembly of any example herein, particularly of example 16, wherein:

the stator is indirectly couplable to the body through a housing;

the drive shaft is indirectly couplable to the adjustment knob through a turbine; and

the electric motor assembly further comprises:

the housing that has a first end couplable to the body and a second end that houses the electric motor, the housing having a fixed angular orientation relative to the body when the first end is coupled to the body; and

the turbine coupled to the drive shaft and operably couplable to the adjustment knob, the turbine having a fixed angular orientation relative to the drive shaft and the adjustment knob, the adjustment knob axially slidable along the turbine when operably coupled to the adjustment knob and in response to rotation of the turbine and the adjustment knob.

Example 18. The electric motor assembly of any example herein, particularly of example 17, wherein the adjustment knob includes two or more axially extending grooves and the turbine includes two or more blades each of which is receivable in and engageable with a corresponding one of the axially extending grooves of the adjustment knob.

Example 19. The electric motor assembly of any example herein, particularly of example 17 or 18, further comprising a clamp to couple the first end to the body.

Example 20. The electric motor assembly of any example herein, particularly of example 19, wherein the clamp to couple the first end to the body comprises a hinged clamshell clamp.

Example 21. The electric motor assembly of any example herein, particularly of any one of examples 17-20, wherein the adjustment knob is axially translatable relative to the body by a translatable distance and a length of the turbine exceeds the translatable distance to accommodate sliding of the adjustment knob relative to the turbine through the entire translatable distance.

Example 22. The electric motor assembly of any example herein, particularly of any one of examples 12-21, further comprising the power supply, wherein the power supply comprises a battery.

Example 23. The electric motor assembly of any example herein, particularly of any one of examples 12-22, further comprising the control device, wherein the control device comprises a potentiometer coupled to the electric motor and a button mountable to the IOL injection device, the button operably coupled to the potentiometer and operable by a user to adjust a rotational speed of the electric motor.

Example 24. The electric motor assembly of any example herein, particularly of example 23, wherein the button comprises a sliding button.

Example 25. The electric motor assembly of any example herein, particularly of any one of examples 12-24, wherein the control device is wirelessly coupled to the electric motor.

Example 26. The electric motor assembly of any example herein, particularly of any one of examples 12-24, wherein the control device is hardwired to the electric motor.

Example 27. The electric motor assembly of any example herein, particularly of any one of examples 12-21, wherein the power supply comprises a phacoemulsification surgical console and the control device comprises a foot pedal of the phacoemulsification surgical console.

Example 28. An intraocular lens (IOL) injection system, comprising:

an IOL injection device, comprising a body, a plunger rod axially translatable relative to the body, and an adjustment knob rotatable and axially translatable relative to the body; and

an electric motor assembly removably coupled to the IOL injection device, the electric motor assembly comprising an electric motor couplable to a power supply and a control device, the electric motor comprising:

a stator; and

a drive shaft;

wherein:

the stator is coupled to an adjustment knob of the IOL injection device with a fixed angular orientation relative to the adjustment knob and the drive shaft is coupled to a plunger rod of the IOL injection device with a fixed angular orientation relative to the plunger rod; or

the stator is coupled to a body of the IOL injection device with a fixed angular orientation relative to the body and the drive shaft is coupled to the adjustment knob with a fixed angular orientation relative to the adjustment knob.

Example 29. The IOL injection system of any example herein, particularly of example 28, wherein:

the stator is coupled to the adjustment knob;

the drive shaft is coupled to the plunger rod;

the adjustment knob threadably engages the body;

operation of the electric motor is effective to rotate the stator and the adjustment knob relative to the plunger rod and the body; and

rotation of the adjustment knob relative to the body is effective to axially translate the plunger rod relative to the body.

Example 30. The IOL injection system of any example herein, particularly of example 28 or 29, wherein:

the plunger rod includes a rod end that defines a slot and the drive shaft includes a shaft end received in the slot; and

the shaft end includes TPU coupled to the slot of the rod end by a friction fit between the TPU and the slot.

Example 31. The IOL injection system of any example herein, particularly of example 28, wherein:

the stator is coupled to the body;

the drive shaft is coupled to the adjustment knob;

the adjustment knob threadably engages the body;

operation of the electric motor is effective to rotate the drive shaft and the adjustment knob relative to the plunger rod and the body; and

rotation of the adjustment knob relative to the body is effective to axially translate the plunger rod relative to the body.

Example 32. The IOL injection system of any example herein, particularly of example 31, wherein:

the stator is indirectly coupled to the body through a housing;

the drive shaft is indirectly coupled to the adjustment knob through a turbine; and

the electric motor assembly further comprises:

the housing that has a first end coupled to the body and a second end that houses the electric motor, the housing having a fixed angular orientation relative to the body when the first end is coupled to the body; and

the turbine coupled to the drive shaft and operably coupled to the adjustment knob, the turbine having a fixed angular orientation relative to the drive shaft and the adjustment knob, the adjustment knob axially slidable along the turbine when operably coupled to the adjustment knob and in response to rotation of the turbine and the adjustment knob.

Example 33. The IOL injection system of any example herein, particularly of example 32, wherein the adjustment knob includes a plurality of axially extending grooves and the turbine includes two or more blades each of which is received in and engaged with a corresponding one of the axially extending grooves of the adjustment knob.

Example 34. The IOL injection system of any example herein, particularly of example 32 or 33, wherein the adjustment knob is axially translatable relative to the body by a translatable distance and a length of the turbine exceeds the translatable distance to accommodate sliding of the adjustment knob relative to the turbine through the entire translatable distance.

Example 35. The IOL injection system of any example herein, particularly of any one of examples 28-34, wherein:

the electric motor assembly further comprises the control device;

the control device comprises a potentiometer coupled to the electric motor and a slidable button mounted to the IOL injection device; and

the slidable button is operably coupled to the potentiometer and is operable by a user to initiate rotation and adjust a rotational speed of the stator or the drive shaft.

Example 36. The IOL injection system of any example herein, particularly of any one of examples 28-35, wherein the electric motor assembly further comprises the power supply and the power supply comprises a battery.

Example 37. The IOL injection system of any example herein, particularly of any one of examples 28-35, wherein the power supply comprises a phacoemulsification surgical console and the control device comprises a foot pedal of the phacoemulsification surgical console.

Example 38. A method to operate an intraocular lens (IOL) injection device, comprising:

removably coupling a stator of an electric motor of an electric motor assembly to an adjustment knob or body of the IOL injection device;

removably coupling a drive shaft of the electric motor to a plunger rod of the IOL injection device or the adjustment knob;

receiving operator input at a control device coupled to the electric motor to initiate rotation between the stator and the drive shaft of the electric motor;

rotating the adjustment knob responsive to the relative rotation between the stator and the drive shaft; and

axially translating the adjustment knob and the plunger rod responsive to rotation of the adjustment knob.

Example 39. The method of any example herein, particularly of example 38, wherein receiving the operator input at the control device comprises receiving input effective to slide a button coupled to a potentiometer of the control device relative to the body.

Example 40. The method of any example herein, particularly of example 38, wherein receiving the operator input at the control device comprises receiving input effective to operate a foot pedal coupled to the electric motor through a phacoemulsification surgical console.

### HYDRAULIC MOTOR EXAMPLE EMBODIMENTS

Example 1. A hydraulic motor assembly removably couplable to an intraocular lens (IOL) injection device, the hydraulic motor assembly comprising:

a hydraulic motor couplable to a fluid supply and a control device, the hydraulic motor comprising:

a stator; and

a rotor;

wherein:

the stator is couplable to an adjustment knob of the IOL injection device with a fixed angular orientation relative to the adjustment knob and the rotor is couplable to a plunger rod of the IOL injection device with a fixed angular orientation relative to the plunger rod; or

the stator is couplable to a body or the plunger rod of the IOL injection device with a fixed angular orientation relative to the body or the plunger rod and the rotor is couplable to the adjustment knob with a fixed angular orientation relative to the adjustment knob.

Example 2. The hydraulic motor assembly of any example herein, particularly of example 1, wherein:

the stator is couplable to the adjustment knob;

the rotor is couplable to the plunger rod;

operation of the hydraulic motor is effective to rotate the stator and the adjustment knob relative to the plunger rod and the body; and

rotation of the adjustment knob relative to the body is effective to axially translate the plunger rod relative to the body.

Example 3. The hydraulic motor assembly of any example herein, particularly of example 2, wherein the plunger rod includes a rod end that defines a slot and the rotor includes a shaft end receivable in the slot.

Example 4. The hydraulic motor assembly of any example herein, particularly of example 3, wherein the shaft end comprises TPU and the shaft end of the rotor is couplable to the slot of the rod end by a friction fit between the TPU and the slot.

Example 5. The hydraulic motor assembly of any example herein, particularly of example 1, wherein:

the stator is couplable to the body or the plunger rod;

the rotor is couplable to the adjustment knob;

operation of the hydraulic motor is effective to rotate the rotor and the adjustment knob relative to the plunger rod and the body; and

rotation of the adjustment knob relative to the body is effective to axially translate the plunger rod relative to the body.

Example 6. The hydraulic motor assembly of any example herein, particularly of example 5, wherein:

the rotor includes a turbine with two or more blades;

the adjustment knob includes two or more axially extending grooves; and

each of the two or more blades is receivable in and engageable with a corresponding one of the axially extending grooves of the adjustment knob.

Example 6A. The hydraulic motor assembly of any example herein, particularly of example 4, wherein:

the rotor is couplable to the adjustment knob through a coupler;

the adjustment knob includes two or more axially extending grooves;

the coupler includes two or more inwardly directed blades, vanes, or ridges that have a complementary shape and size to the two or more axially extending grooves of the adjustment knob;

each of the two or more blades, vanes, or ridges is receivable in and engageable with a corresponding one of the axially extending grooves of the adjustment knob.

Example 7. The hydraulic motor assembly of any example herein, particularly of example 5 or 6, further comprising a clamp to couple the stator to the body.

Example 8. The hydraulic motor assembly of any example herein, particularly of example 7, wherein the clamp to couple the stator to the body comprises a hinged clamshell clamp.

Example 9. The hydraulic motor assembly of any example herein, particularly of any one of examples 6-8, wherein the adjustment knob is axially translatable relative to the body by a translatable distance and a length of the turbine exceeds the translatable distance to accommodate sliding of the adjustment knob relative to the turbine through the entire translatable distance.

Example 10. The hydraulic motor assembly of any example herein, particularly of any one of examples 1-9, wherein the fluid supply comprises a phacoemulsification surgical console and the control device comprises a foot pedal of the phacoemulsification surgical console.

Example 11. The hydraulic motor assembly of any example herein, particularly of example 10, further comprising a cassette coupled between the fluid supply and the hydraulic motor, the cassette comprising a pump.

Example 12. An intraocular lens (IOL) injection system, comprising:

an IOL injection device, comprising a body, a plunger rod axially translatable relative to the body, and an adjustment knob rotatable and axially translatable relative to the body; and

a hydraulic motor assembly removably coupled to the IOL injection device, the hydraulic motor assembly comprising a hydraulic motor couplable to a fluid supply and a control device, the hydraulic motor comprising:

a stator; and

a rotor;

wherein:

the stator is coupled to the adjustment knob or the plunger rod of the IOL injection device with a fixed angular orientation relative to the adjustment knob or the plunger rod and the rotor is coupled to the plunger rod of the IOL injection device with a fixed angular orientation relative to the plunger rod; or

the stator is coupled to the body of the IOL injection device with a fixed angular orientation relative to the body and the rotor is coupled to the adjustment knob with a fixed angular orientation relative to the adjustment knob.

Example 13. The IOL injection system of any example herein, particularly of example 12, wherein:

the stator is coupled to the adjustment knob;

the rotor is coupled to the plunger rod;

the adjustment knob threadably engages the body;

operation of the hydraulic motor is effective to rotate the stator and the adjustment knob relative to the plunger rod and the body; and

rotation of the adjustment knob relative to the body is effective to axially translate the plunger rod relative to the body.

Example 14. The IOL injection system of any example herein, particularly of example 12 or 13, wherein:

the plunger rod includes a rod end that defines a slot and the rotor includes a shaft end received in the slot; and

the shaft end comprises TPU coupled to the slot of the rod end by a friction fit between the TPU and the slot.

Example 15. The IOL injection system of any example herein, particularly of example 12, wherein:

the stator is coupled to the body or the plunger rod;

the rotor is coupled to the adjustment knob;

the adjustment knob threadably engages the body;

operation of the hydraulic motor is effective to rotate the rotor and the adjustment knob relative to the plunger rod and the body; and

rotation of the adjustment knob relative to the body is effective to axially translate the plunger rod relative to the body.

Example 16. The IOL injection system of any example herein, particularly of example 12 or 15, wherein:

the rotor includes a turbine with two or more blades;

the adjustment knob includes two or more axially extending grooves; and

each of the two or more blades is received in and engages with a corresponding one of the axially extending grooves of the adjustment knob.

Example 16A. The IOL injection system of any example herein, particularly of example 12, wherein:

the rotor is coupled to the adjustment knob through a coupler;

the adjustment knob includes two or more axially extending grooves;

the coupler includes two or more inwardly directed blades, vanes, or ridges that have a complementary shape and size to the two or more axially extending grooves of the adjustment knob;

each of the two or more blades, vanes, or ridges is receivable in and engageable with a corresponding one of the axially extending grooves of the adjustment knob.

Example 17. The IOL injection system of any example herein, particularly of example 16, wherein the adjustment knob is axially translatable relative to the body by a translatable distance and a length of the turbine exceeds the translatable distance to accommodate sliding of the adjustment knob relative to the turbine through the entire translatable distance.

Example 18. The IOL injection system of any example herein, particularly of any one of examples 12-17, wherein the fluid supply comprises a phacoemulsification surgical console and the control device comprises a foot pedal of the phacoemulsification surgical console.

Example 19. The hydraulic motor assembly of any example herein, particularly of any one of examples 12-18, further comprising a cassette coupled between the fluid supply and the hydraulic motor, the cassette comprising a pump.

Example 20. A method to operate an intraocular lens (IOL) injection device, comprising:

removably coupling a stator of a hydraulic motor of a hydraulic motor assembly to an adjustment knob, body, or plunger rod of the IOL injection device;

removably coupling a rotor of the hydraulic motor to the plunger rod or the adjustment knob;

receiving operator input at a control device coupled to the hydraulic motor to initiate relative rotation between the stator and the rotor of the hydraulic motor;

rotating the adjustment knob responsive to the relative rotation between the stator and the rotor; and

axially translating the adjustment knob and the plunger rod responsive to rotation of the adjustment knob.

Example 21. The method of any example herein, particularly of example 20, wherein receiving the operator input at the control device comprises receiving input effective to operate a foot pedal coupled to the hydraulic motor through a phacoemulsification surgical console.

Example 22. The method of any example herein, particularly of example 20, further comprising, prior to receiving the operator input, fluidly coupling the hydraulic motor to a fluid supply, the fluid supply comprising a phacoemulsification surgical console.

Example 23. The method of any example herein, particularly of example 21 or 22, wherein fluidly coupling the hydraulic motor to the fluid supply comprises coupling input and output fluid ports of the hydraulic motor assembly to a disposable cassette coupled to the phacoemulsification surgical console.

### Example Set 2

Example 1. An assembly operable with an IOL injection device, the assembly comprising:

an electric motor configured to generate rotational motion, the electric motor comprising a stator and a drive shaft, the stator or the drive shaft removably couplable to an adjustment knob of the IOL injection device; and

a control device communicatively coupled to the electric motor and configured to control operation of the electric motor;

wherein the stator or the drive shaft is configured to rotate the adjustment knob relative to a body and a plunger rod of the IOL injection device in response to the rotational motion.

Example 2. The assembly of any example herein, particularly of example 1, wherein the stator is couplable to the adjustment knob of the IOL injection device with a fixed angular orientation relative to the adjustment knob and the drive shaft is couplable to the plunger rod of the IOL injection device with a fixed angular orientation relative to the plunger rod.

Example 3. The assembly of any example herein, particularly of example 1, wherein the stator is couplable to the body of the IOL injection device with a fixed angular orientation relative to the body and the drive shaft is couplable to the adjustment knob of the IOL injection device with a fixed angular orientation relative to the adjustment knob.

Example 4. The assembly of any example herein, particularly of any one of examples 1-3, wherein the control device is configured to provide variable rotational speed control of the electric motor.

Example 5. The assembly of any example herein, particularly of any one of examples 1-4, wherein the control device is configured to provide bidirectional rotation control of the electric motor.

Example 6. The assembly of any example herein, particularly of any one of examples 1 and 4-5, further comprising a flex shaft cable having a first end coupled to the drive shaft of the electric motor and a second end couplable to the adjustable knob of the IOL injection device, the flex shaft cable configured to transmit the rotational motion from the electric motor to the adjustment knob.

Example 7. The assembly of any example herein, particularly of example 6, wherein the control device comprises a foot pedal configured to receive user input to control at least one of initiation of rotation of the electric motor, termination of rotation of the electric motor, a rotational speed of the electric motor, or a rotational direction of the electric motor.

Example 8. The assembly of any example herein, particularly of any one of examples 6-7, wherein the electric motor is part of a standalone unit with an integrated power supply.

Example 9. The assembly of any example herein, particularly of any one of examples 6-7, wherein the electric motor is part of a phacoemulsification surgical console.

Example 10. The assembly of any example herein, particularly of example 9, wherein the electric motor selectively controls operation of the adjustment knob of the IOL injection device or of a peristaltic pump of the phacoemulsification surgical console.

Example 11. An IOL injection system, comprising:

the assembly of any example herein, particularly of any one of examples 1-10; and

the IOL injection device comprising the body, the plunger rod axially translatable relative to the body, and the adjustment knob rotatable and axially translatable relative to the body.

Example 12. An assembly operable with an IOL injection device, the assembly comprising:

a flex shaft cable comprising a first end couplable to an electric motor configured to generate rotational motion and a second end couplable to an adjustment knob of the IOL injection device;

a first connector at the first end of the flex shaft cable and configured to couple the first end of the flex shaft cable to the electric motor; and

a second connector at the second end of the flex shaft cable and configured to couple the second end of the flex shaft cable to the adjustment knob;

wherein the flex shaft cable is configured to transmit the rotational motion from the electric motor and the second connector is configured to rotate the adjustment knob in response to the rotational motion transmitted through the flex shaft cable.

Example 13. The assembly of any example herein, particularly of example 12, wherein the flex shaft cable comprises a sterilizable outer sheath.

Example 14. The assembly of any example herein, particularly of any one of examples 12-13, wherein the flex shaft cable is sterilizable.

Example 15. The assembly of any example herein, particularly of any one of examples 12-14, wherein the first connector is configured to detachably couple the first end of the flex shaft cable to the electric motor.

Example 16. The assembly of any example herein, particularly of any one of examples 12-15, wherein the second connector is configured to detachably couple the second end of the flex shaft cable to the adjustment knob of the IOL injection device.

Example 17. A method of delivering an intraocular lens (IOL) comprising:

attaching a connector to an adjustment knob of an IOL injection device, wherein the connector is coupled to a flex shaft cable;

activating an electric motor mechanically coupled to the flex shaft cable to generate rotational motion;

transmitting the rotational motion from the electric motor through the flex shaft cable and the connector to the adjustment knob; and

rotating the adjustment knob of the intraocular lens injection device to advance an IOL relative to the IOL injection device.

Example 18. The method of any example herein, particularly of example 17, further comprising controlling the electric motor via a foot pedal.

Example 19. The method of any example herein, particularly of example 18, wherein controlling the electric motor includes controlling a rotational speed or rotational direction of the electric motor.

Example 20. The method of any example herein, particularly of any one of examples 17-19, further comprising sterilizing the flex shaft cable and the connector prior to the attaching.

What is claimed is:

1. An assembly operable with an intraocular lens (IOL) injection device, the assembly comprising:
an electric motor configured to generate rotational motion, the electric motor comprising a stator and a drive shaft, the stator or the drive shaft removably couplable to an adjustment knob of the IOL injection device; and
a control device communicatively coupled to the electric motor and configured to control operation of the electric motor;
wherein the stator or the drive shaft is configured to rotate the adjustment knob relative to a body and a plunger rod of the IOL injection device in response to the rotational motion.

2. The assembly of claim 1, wherein the stator is couplable to the adjustment knob of the IOL injection device with a fixed angular orientation relative to the adjustment knob and the drive shaft is couplable to the plunger rod of the IOL injection device with a fixed angular orientation relative to the plunger rod.

3. The assembly of claim 1, wherein the stator is couplable to the body of the IOL injection device with a fixed angular orientation relative to the body and the drive shaft is couplable to the adjustment knob of the IOL injection device with a fixed angular orientation relative to the adjustment knob.

4. The assembly of claim 1, wherein the control device is configured to provide variable rotational speed control of the electric motor.

5. The assembly of claim 1, wherein the control device is configured to provide bidirectional rotation control of the electric motor.

6. The assembly of claim 1, further comprising a flex shaft cable having a first end coupled to the drive shaft of the electric motor and a second end couplable to the adjustable knob of the IOL injection device, the flex shaft cable configured to transmit the rotational motion from the electric motor to the adjustment knob.

7. The assembly of claim 6, wherein the control device comprises a foot pedal configured to receive user input to control at least one of initiation of rotation of the electric motor, termination of rotation of the electric motor, a rotational speed of the electric motor, or a rotational direction of the electric motor.

8. The assembly of claim 6, wherein the electric motor is part of a standalone unit with an integrated power supply.

9. The assembly of claim 6, wherein the electric motor is part of a phacoemulsification surgical console.

10. The assembly of claim 9, wherein the electric motor selectively controls operation of the adjustment knob of the IOL injection device or of a peristaltic pump of the phacoemulsification surgical console.

11. An IOL injection system, comprising:
the assembly of claim 1; and
the IOL injection device comprising the body, the plunger rod axially translatable relative to the body, and the adjustment knob rotatable and axially translatable relative to the body.

12. An assembly operable with an intraocular lens (IOL) injection device, the assembly comprising:
a flex shaft cable comprising a first end couplable to an electric motor configured to generate rotational motion and a second end couplable to an adjustment knob of the IOL injection device;
a first connector at the first end of the flex shaft cable and configured to couple the first end of the flex shaft cable to the electric motor; and
a second connector at the second end of the flex shaft cable and configured to couple the second end of the flex shaft cable to the adjustment knob;
wherein the flex shaft cable is configured to transmit the rotational motion from the electric motor and the second connector is configured to rotate the adjustment knob in response to the rotational motion transmitted through the flex shaft cable.

13. The assembly of claim 12, wherein the flex shaft cable comprises a sterilizable outer sheath.

14. The assembly of claim 12, wherein the flex shaft cable is sterilizable.

15. The assembly of claim 12, wherein the first connector is configured to detachably couple the first end of the flex shaft cable to the electric motor.

16. The assembly of claim 12, wherein the second connector is configured to detachably couple the second end of the flex shaft cable to the adjustment knob of the IOL injection device.

17. A method of delivering an intraocular lens (IOL) comprising:
attaching a connector to an adjustment knob of an IOL injection device, wherein the connector is coupled to a flex shaft cable;
activating an electric motor mechanically coupled to the flex shaft cable to generate rotational motion;
transmitting the rotational motion from the electric motor through the flex shaft cable and the connector to the adjustment knob; and
rotating the adjustment knob of the IOL injection device to advance an IOL relative to the IOL injection device.

18. The method of claim 17, further comprising controlling the electric motor via a foot pedal.

19. The method of claim 18, wherein controlling the electric motor includes controlling a rotational speed or rotational direction of the electric motor.

20. The method of claim 17, further comprising sterilizing the flex shaft cable and the connector prior to the attaching.
